(19) United States
(12) Patent Application Publication
(10) Pub. No.: US 2023/0158187 A1
(43) Pub. Date: May 25, 2023

(54) METHODS, SYSTEMS, AND APPARATUSES FOR DISINFECTING AND SANITIZING MATERIALS

(71) Applicant: John-Paul BONANSINGA, San Antonio, TX (US)

(72) Inventor: John-Paul BONANSINGA, San Antonio, TX (US)

(21) Appl. No.: 18/092,701

(22) Filed: Jan. 3, 2023

Related U.S. Application Data
(63) Continuation-in-part of application No. PCT/US2021/040205, filed on Jul. 1, 2021.
(60) Provisional application No. 63/047,138, filed on Jul. 1, 2020.

(51) Int. Cl. A61L 2/10

(52) U.S. Cl. CPC A61L 2/10; A61L 2202/122; A61L 2202/14

(57) ABSTRACT

An apparatus includes a housing defining a reactor chamber and an access door configured to transition between an open configuration to allow access to reactor chamber from outside the interior chamber to a closed configuration to prevent access to the reactor chamber from outside the reactor chamber, a holder configured to support within the reactor chamber an object to be disinfected, a plurality of electromagnetic radiation (EMR) emitters disposed within the reactor chamber and configured to emit electromagnetic radiation to treat the object, a plurality of sensors disposed within the housing and configured to detect radiation intensity, and a controller configured to calculate a time period to meet a predefined radiation exposure threshold for a treatment cycle based on the radiation intensity, the controller configured to send a signal to identify completion of the treatment cycle in response to completion of the time period.

### CROSS-REFERENCE TO RELATED APPLICATIONS

This application is a continuation-in-part of International Patent Application No. PCT/US2021/040205, entitled “DEVICES, SYSTEMS, AND METHODS FOR DISINFECTING AND SANITIZING MATERIALS,” filed Jul. 1, 2021, which claims priority to and the benefit of U.S. Provisional Patent Application No. 63/047,138, entitled “DEVICES, SYSTEMS, AND METHODS FOR DISINFECTING AND SANITIZING MATERIALS,” filed Jul. 1, 2020, each disclosure of which is incorporated herein in reference in its entirety.

### TECHNICAL FIELD

The embodiments described herein relate generally to methods, systems, and apparatuses for sterilizing, disinfecting, and/or sanitizing articles and materials with electromagnetic radiation (EMR).

### BACKGROUND

Personal protective equipment (PPE) (e.g., gloves, masks, scrubs, filters, eyewear, shoe-covers, drapes, etc.) is used to prevent the spread of disease and infection. These items are in greatest demand in health care settings such as hospitals, doctor's offices, and clinical labels. When used properly, PPE acts as a barrier between infectious materials, such as viruses and bacteria, and the skin, mouth, nose, and eyes. This barrier prevents passage of these and other infectious agents into the body. PPE also protects patients from exposure to harmful agents, particularly at higher risk for contracting infections and/or undergoing a surgical procedure. PPE is often used once and discarded which generates significant quantities of waste when PPE is used often.

Certain other high-use objects in medical settings (e.g., stethoscopes, mobile phones, pens, clipboards, non-expendable personal exam tools, etc.) can also be contaminated during standard use. Standard procedure for treating these high-use object can include elective and/or periodic wiping with antimicrobial wipes. These wipes are often not in contact with the surface contaminants for a desired amount of time and can result in sublethal doses that can cause contaminants to become resistant to antimicrobial wipes.

In order to reduce the waste generated from PPE/high-use objects, sterilization, disinfection, and/or sanitation can be used. Current methods of sterilization and/or disinfection of PPE/high-use objects can be time consuming and have the risk of destroying and/or damaging the PPE/high-use objects and, in certain circumstances, can leave the PPE/high-use objects contaminated or no longer providing sufficient protection against contaminants. Certain methods utilize heat to kill bacteria on PPE/high-use objects, but this method may not be able to target all types of bacteria and/or viruses without damaging the PPE with high heat. Furthermore, the PPE/high-use objects may be run in disinfection cycles that sanitize the PPE/high-use objects for a predetermined amount of time, which may be longer than necessary. Longer than necessary PPE/high-use objects sanitizing times may be cumbersome for sanitizing a large number of PPE/high-use objects. Thus there is a need for sanitizing and/or disinfecting PPE/high-use objects in a manner that reduces the amount of time to sanitize and/or disinfect and doesn't damage the PPE/high-use objects in the process.

### SUMMARY

In one embodiment, an apparatus includes a housing defining a reactor chamber and an access door configured to transition between an open configuration to allow access to reactor chamber from outside the interior chamber to a closed configuration to prevent access to the reactor chamber from outside the reactor chamber, a holder configured to support within the reactor chamber an object to be disinfected, a plurality of electromagnetic radiation (EMR) emitters disposed within the reactor chamber and configured to emit EMR to treat the object, the plurality of EMR emitters including a first EMR emitter and a second EMR emitter, a plurality of sensors disposed within the housing and configured to detect radiation intensity, the plurality of sensors including a first sensor configured to detect first pass radiation from the first emitter and not from the second emitter to measure radiation intensity emitted by the first EMR emitter, and a second sensor configured to measure first pass radiation from the second emitter and not from the first emitter to identify radiation intensity emitted by the second EMR emitter, and a controller configured to calculate a time period to meet a predefined radiation exposure threshold for a treatment cycle based on the lower of the first pass radiation detected by the first sensor and the first pass radiation measured by the second sensor, the controller configured to send a signal to identify completion of the treatment cycle in response to completion of the time period.

In one embodiment, a method to determine a time period to operate a radiation treatment cycle for an object disposed within a reactor chamber of a housing, the reactor chamber including a plurality of electromagnetic radiation (EMR) emitters configured to emit radiation to treat the object, the housing including a plurality of sensors configured to measure radiation intensity. The method includes receiving from a first sensor from the plurality of sensors a radiation intensity value representative of first pass radiation emitted within the reactor chamber by a first EMR emitter from the plurality of EMR emitters, receiving from a second sensor from the plurality of sensors a radiation intensity value representative of first pass radiation emitted within the reactor chamber by a second EMR emitter from the plurality of EMR emitters, identifying a lowest output EMR emitter from the plurality of EMR emitters based on the lesser of the radiation intensity value received from the first sensor and the radiation intensity value received from the second sensor to define a minimum collective output for the plurality of EMR emitters, and calculating a time period to deliver a predefined minimum radiation exposure threshold of radiation to an area on the object expected to receive the least radiation exposure per unit time relative to the remainder of the object, based on (1) the minimum collective output, (2) a distance associated with first pass radiation provided by one EMR emitter from the plurality of EMR emitters, and (3) a distance and reflectivity factor associated with second pass radiation provided by one EMR emitter from the plurality of EMR emitters.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a schematic diagram of an EMR system, according to an embodiment.

FIG. 2 is a schematic diagram of a controller, according to an embodiment.

FIG. 3A is a method of treating an object, according to an embodiment.

FIG. 3B is a method of treating an object, according to an embodiment.

FIG. 4A depicts a front view of a reactor chamber with first pass EMR dispersion on an object shown, according to an embodiment.

FIG. 4B depicts a front view of a reactor chamber with first pass and close second pass EMR dispersion shown, according to an embodiment.

FIG. 4C depicts a front view of a reactor chamber with first pass and close second pass EMR dispersion on an object shown, according to an embodiment.

FIG. 4D depicts a front view of a reactor chamber with EMR rays on an object shown, according to an embodiment.

FIG. 5A depicts a perspective view of a front of an EMR system, according to an embodiment.

FIG. 5B depicts a perspective view of the back of the EMR system of FIG. 5A.

FIG. 5C depicts a front view of the EMR system of FIG. 5A.

FIG. 6A depicts a front view of a reactor chamber of an EMR system, according to an embodiment.

FIG. 6B depicts a detailed view of a sensor location relative to the reactor chamber of FIG. 6A,

FIG. 7A depicts a perspective view of an EMR system with additional hardware in an open state, according to an embodiment.

FIG. 7B depicts a perspective view of the EMR system of FIG. 7A in a closed state.

FIG. 8 depicts an input/output device of an EMR system, according to an embodiment.

FIG. 9 depicts a top view of an input/output device, according to an embodiment.

FIG. 10A depicts a side view of a single EMR emitter configuration, according to an embodiment.

FIG. 10B depicts a perspective view of the single EMR emitter configuration of FIG. 10A

FIG. 10C depicts a side view of a single EMR emitter configuration with additional reflectors, according to an embodiment.

FIG. 11A depicts a front view of a reactor chamber with dispersion beams, according to an embodiment.

FIG. 11B depicts the reactor chamber of FIG. 11A with a large object within.

FIG. 11C depicts a perspective view of the reactor chamber of FIG. 11A with a small object within.

FIG. 11D depicts a perspective view of the reactor chamber of FIG. 11A with a large object within.

FIG. 12A depicts a sensor mounted on the housing of an EMR system, according to an embodiment.

FIG. 12B depicts the internal portion of the sensor of FIG. 12A.

### DETAILED DESCRIPTION

In some implementations, an apparatus for treating PPE (e.g., gloves, masks, scrubs, filters, eyewear, shoe-covers, etc.) and/or high-use object includes a housing defining a reactor chamber and an access door. In some embodiments, the access door transitions between an open configuration to allow access to the reactor chamber from outside the reactor chamber to a closed configuration to prevent access to the reactor chamber from outside the reactor chamber. Within the reactor chamber, the apparatus includes a holder configured to support an object to be disinfected and a plurality of electromagnetic radiation (EMR) emitters. The EMR emitters emit EMR to treat the object. In some embodiments, the plurality of EMR emitters include a first EMR emitter and a second EMR emitter. A plurality of sensors can be disposed within the housing and configured to detect radiation intensity. In some embodiments, the plurality of sensors includes a first EMR sensor configured to detect first pass radiation from the first emitter and not from the second emitter to measure radiation intensity emitted by the first EMR emitter, and a second sensor configured to measure first pass radiation from the second emitter and not from the first emitter to identify radiation intensity emitted by the second EMR emitter. The apparatus further includes a controller configured to calculate a time period to meet a predefined radiation exposure threshold for a treatment cycle based on the lower of the first pass radiation detected by the first second and the first pass radiation measured by the second sensor, the controller configured to send a signal to identify completion of the treatment cycle in response to completion of the time period.

Some techniques described herein allow for objects to be treated in an EMR system thoroughly and without damaging the object in a decreased amount of time when compared with current methods. Some techniques described herein use EMR to treat the object. This allows for the object to be treated without damaging the object. To decrease the amount of time, some of the techniques described herein include determining the intensity of EMR on the object within the EMR system and based on the intensity, determining a minimum amount of time needed to fully treat the object. This allows for objects to be treated efficiently by determining a treatment time for the specific object and object orientation. Efficiently treating objects allows for more objects to be treated and decreases the energy draw of the treatment process.

Some techniques described herein reduce the likelihood of overexposing an object, which can damage the object. Some techniques described herein utilize sensors to determine current and/or real-time radiation intensity levels of EMR emitters. Some techniques described herein determine radiation exposure on each surface of an object. Some techniques described herein determine reflections of the EMR and reflectivity of surfaces. The radiation exposure on each surface of the object, reflections, and the radiation intensity levels of the EMR emitters can be used to determine a minimum time for sufficiently treating an entire object. In some embodiments, the minimum time can be determined based on the lowest radiation exposure and/or based on the lowest emitting EMR emitter. The minimum time allows for the object to be fully treated and reduces the likelihood of overexposure.

As used herein, the term “treat,” “treating,” and the like can refer to any combination of sanitizing, disinfecting, and/or sterilizing. In some embodiments, treating an object can include delivering a dose of EMR, to all surfaces of the object, that is lethal to bacteria and/or viruses.

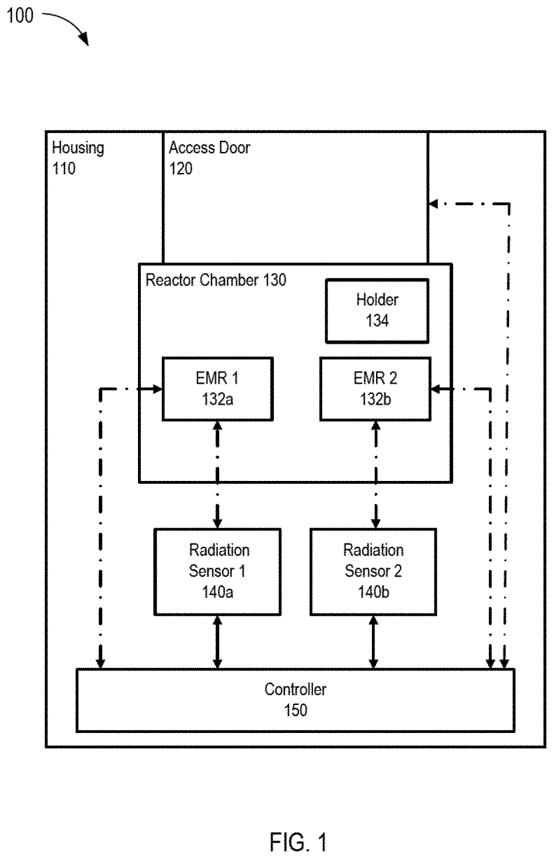

FIG. 1 is a schematic diagram of an EMR system 100, according to an embodiment. The EMR system 100 is configured to receive and treat (e.g., sterilize, disinfect, and/or sanitize) an object via EMR. In some embodiments, the EMR is ultraviolet (UV) C radiation. The EMR system 100 can be a stand-alone system or, in some embodiments, can be a part of a larger treatment system. In some embodiments, the EMR system 100 can be a portable device configured to be relocated for use. The EMR system 100 includes a housing 110, an access door 120, a reactor chamber 130 including a holder 134 and EMR emitters (132a and 132b), radiation sensors (140a and 140b), and a controller 150.

In some embodiments, the EMR system 100 is electrically coupled to a power source (e.g., battery, generator, power supply, etc.). The power source can be located on the EMR system 100 or can be located externally to the EMR system 100 and electrically coupled thereto. For example, the EMR system 100 can include an internal power source such as battery, generator, solar array, and/or the like and/or can be electrically coupled to an external power source such as a generator, outlet power, etc.

The housing 110 supports and protects the components of the EMR system 100. In some embodiments, the housing 110 can be formed or coated with a sanitary coating. In some embodiments, at least a portion of the housing 110 is formed of aluminum. In some embodiments, at least a portion of the housing 110 is coated in a fingerprint proof hard coating with ion silver anti-microbial properties. The hard coating can prevent the housing 110 from damage and/or to reduce microbial growth on the housing 110. This also decreases the potential sources of contamination so that the EMR system 100 does not further generate contaminants outside of the object to be treated. The housing 110 may include supports or other features that allow for the EMR system 110 to be placed on a surface for usage. In some embodiments, the housing 110 can be a unitary body or can be formed separately of multiple components (e.g., top surface, left surface, right surface, bottom surface, etc.) and then coupled together. In some embodiments, the housing 110 can include access panels and/or removable components for accessing components of the EMR system 100 for use and/or for maintenance. The housing 110 defines the reactor chamber 130, which is a cavity within the housing 110.

The housing 110 includes and/or is coupled to the access door 120 that is configurable between an open position and a closed position relative to the reactor chamber 130. The access door 120 can be a door, a hatch, a press-fit cover, a screw-on cover, a sliding cover, a series of covers, or any other feature configured to provide and limit access to the reactor chamber 130. As another example, in some embodiments, at least a portion of the reactor chamber 130 may be movable, such that it may be at least partially removed from the housing 110 to provide access and allow a user to introduce an object to be treated, and then reintroduced to the housing 110 for a treatment cycle. In such embodiments, for example, the reactor chamber 130 can slide into and out of the housing, like a drawer. In the open position, the access door 120 allows for the reactor chamber 130 to be accessed from outside the reactor chamber 130. For example, in the open position, a user can place an object in the reactor chamber 130. In the closed position, the access door 120 prevents the reactor chamber 130 from being accessed from outside the reactor chamber 130. Additionally, in the closed position, the access door 120 forms an EMR seal with the housing 110 so that EMR within the reactor chamber 130 does not escape from the reactor chamber 130. This protects the user from the EMR and allows the user to be in the same area as the EMR system 100 during operation. The housing 110 and the access door 120 may have significant overlap in the closed position to allow for additional protection from EMR.

In some embodiments, the access door 120 and the housing 110 are coupled via a fastener (e.g., hinge, clips, pin, etc.). In some embodiments, the access door 120 and the housing 110 are coupled via a press fit or a contact fit. For example, the access door 120 may be placed over the reactor chamber 130. In some embodiments, the access door 120 is locked in place when in the closed position. In some embodiments, the access door 120 is only locked during operation of the EMR system 100. In some embodiments, the access doors 120 and/or the housing 110 includes a sensor that detects the position of the access door 120. In some embodiments, the position indicated by the sensor can affect the operation of the EMR system 100. For example, if the EMR system 100 is operating and the sensor detects the position of the access door 120 is open (or e.g., anything less than fully closed), then the EMR system 100 can cease operation to protect the user from EMR. In some embodiments, the access door 120 includes a magnetic switch that prevents the access door 120 from being opened during operation of the EMR system 100. The inside of the access door 120 (e.g., side facing the reactor chamber 130 in the closed position) can be formed of a reflective material and/or can be coated with a reflective material as to reflect EMR. In some embodiments, the reflective material is polytetrafluoroethylene (PTFE). In some embodiments, the reflective material is less than about 0.06 inches thick. In some embodiments, the reflective material is about 0.020 inches thick.

The reactor chamber 130 is configured to reflect EMR onto an object that is located within the reactor chamber 130. Disposed within the reactor chamber 130 are the holder 134 and EMR emitters (132a and 132b). In the embodiment shown in FIG. 1, the reactor chamber 130 includes one holder 134 but, in some embodiments, the reactor chamber 130 can include any number (e.g., 1, 2, 3, 4, 5, 6, 7, 8, 9, 10, 20, 50, 100, etc.) of holders 134. In some embodiments, the holders 134 can be located within an allowable region of the reactor chamber 130. The allowable region can correspond to the portions of the reactor chamber 130 that can provide sufficient amount of EMR to the object located on the holder 134.

In the embodiment shown in FIG. 1, the reactor chamber 130 includes a first EMR emitter 132a and a second EMR emitter 132b but the reactor chamber 130 can include any number of EMR emitters. In some embodiments, the EMR emitters (132a and 132b) are located within the housing 110 but separated from the reactor chamber 130 by a transparent panel (not shown). The EMR emitters (132a and 132b) can be located symmetrically within the reactor chamber 130. In some embodiments, the locations of the EMR emitters (132a and 132b) within the reactor chamber 130 can be reconfigured. In some embodiments, the EMR emitter (132a and 132b) can be selectively switched on and off to alter the effective EMR emitter (132a and 132b) configuration within the reactor chamber 130. The reactor chamber 130 is configured to expose all surfaces of the object simultaneously to EMR.

The reactor chamber 130 includes walls that are configured to reflect EMR. The walls can be coated and/or formed of a material that reflects EMR. In some embodiments, the walls reflect more than about 85% of EMR. In some embodiments, the walls comprise PTFE, other fluoropolymer (e.g., E-PTFE, D-PTFE, etc.), sputtered glass, aluminum sputtered glass, and/or other material(s) that can reflect EMR. In some embodiments, the reflective coating is less than about 1.5 mm thick. The reflective coating allows for both heat generated by EMR emitters (132a and 132b) to dissipate and for EMR to reflect back within the chamber and increase the intensity and efficiency of the EMR system 100. Reflecting and dissipating heat allows the EMR system 100 to avoid the use of fans and/or cooling units which may introduce fluid droplets onto the object being disinfected. The reactor chamber 130 can include rounded edges or other features (e.g., reflectors, angled portions, etc.) configured to reflect EMR in a particular direction, such as towards an object or a location in the reactor chamber 130. In some embodiments, the walls can be configured to such that at least 50% of EMR emitter (132a and 132b) output is absorbed object to be treated. For example, the walls are configured (e.g., angled, asymmetric reactor chamber, and the like) such that the total amount of EMR absorbed by the object is at least about 50% of the total EMR emitted by the EMR emitters (132a and 132b). In some implementations, the system can be configured with reflective surfaces sufficient to achieve at least 50% efficiency of actual EMR output redirected towards the centroid of the space defined by the interior surfaces. In some such implementations, the walls are configured such that the time duration to achieve such efficiency is minimized. For example, the walls can absorb a portion of the EMR and/or can be shaped such that additional and/or fewer reflections redirect and absorb the EMR. The walls can be configured to minimize and/or reduce the amount of EMR absorbed by the walls and/or the holders 134 to increase the efficiency of the EMR system 100. In some embodiments, the reactor chamber 130 can include mounting hardware such as pegs, holes, slots, and/or the like for mounting the holder 134. In some embodiments, the reactor chamber 130 can include multiple configurations of mounting hardware to allow for the holder 134 to be repositioned within the reactor chamber 130. In some embodiments, the mounting hardware can include sensors (not shown) to determine if an object is supported by the holder 134.

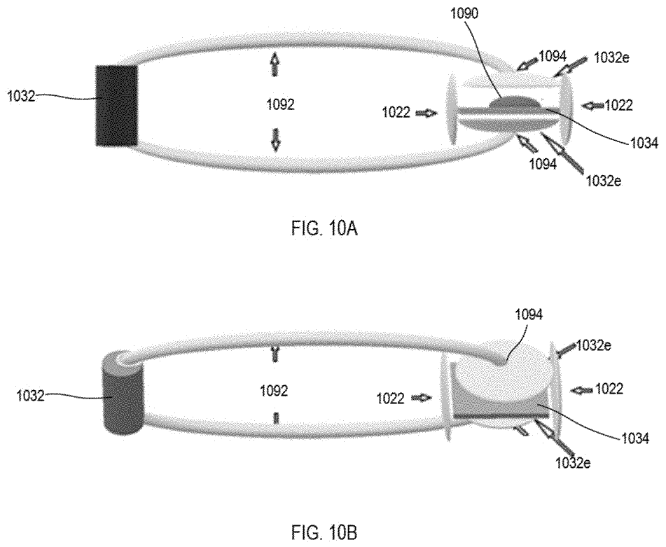

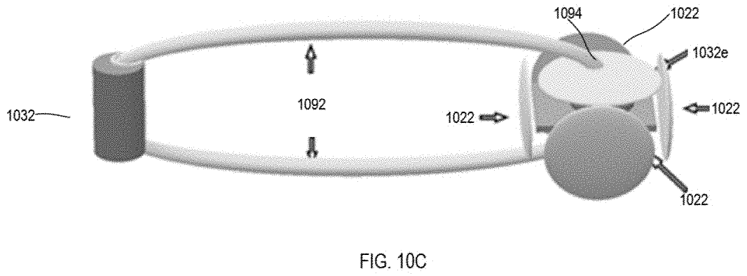

The first EMR emitter 132a and the second EMR emitter 132b are EMR emitters within the reactor chamber 130. In some embodiments, the reactor chamber 130 can include additional EMR emitters or just one EMR emitter. For example, a single EMR emitter configuration can include fiberoptics that direct EMR uniformly across an object during treatment (e.g., as depicted in FIGS. 10A-10C). In some embodiments, the EMR emitters (132a and 132b) are located within the housing 110 behind a layer of material configured to transmit high amount EMR. The EMR emitters (132a and 132b) can be UV-C bulbs. In some embodiments, the EMR emitters (132a and 132b) can include low pressure mercury bulbs, high pressure mercury bulbs, doped mercury bulbs, xenon bulbs, light emitting diodes, and other EMR sources. In some embodiments, the EMR emitters (132a and 132b) can include UV-A and/or UV-B bulbs. The EMR emitters (132a and 132b) can be tube bulbs, round bulbs, and/or other bulb shape configured to emit EMR in multiple directions. In some embodiments, the EMR emitters (132a and 132b) are compact fluorescent lamps. In some embodiments, the EMR emitters (132a and 132b) are gas filled bulbs. In some embodiments, the EMR emitters (132a and 132b) have beam angles of 360 degrees. In some embodiments, the EMR emitters (132a and 132b) can include a combination of bulbs. In some embodiments, the EMR emitters (132a and 132b) are operated by drivers that are configured to run the EMR emitters (132a and 132b).

The holder 134 is a feature of the reactor chamber 130 configured to position an object (not shown) to be treated within the reactor chamber 130. The holder 134 may be a shelf, a hook, stand, bar, loop, ring, or other feature configured to hold, balance, suspend, etc. an object within the reactor chamber 130. In some embodiments, the desired holder 134 is a flat surface (or at least a top surface being flat). The holder 134 can couple to mounting hardware located in the reactor chamber 130. In some embodiments, the reactor chamber 130 can include multiple holders 134. In some embodiments, the holder 134 is formed of a material capable of transmitting EMR. In some embodiments, the holder 134 is formed of a glass and/or an optical grade polymer. In some embodiments, the holder 134 is formed of glass quartz. In some embodiments, the holder 134 transmits more than about 92% of EMR. In some embodiments, the holder 134 is formed of linear low density polyethylene.

The radiation sensors (140a and 140b) are EMR sensors configured to determine the radiation intensity within the reactor chamber 130. Using the radiation sensors (140a and 140b) is advantageous as electronic components, such as bulbs, do not operate consistently and at 100% efficiency and operating conditions may be different. Furthermore, electronic components can degrade over time and projected measurements may become inaccurate over time. Thus, real-time measurements from the radiation sensors (140a and 140b) can provide accurate input data for determining treatment conditions.

In some embodiments, the radiation sensors (140a and 140b) include a UV-C sensor and/or photodiode. In some embodiments, the radiation sensors (140a and 140b) can include operation amplifiers to boost signal and reduce noise/interference. The radiation sensors (140a and 140b) provide real-time performance measurements of each EMR emitter (132a and 132b). The measurements from the radiation sensors (140a and 140b) allow for the EMR system 100 to keep track of the EMR emitters (132a and 132b) and adjust treatment strategies based on the measurements. In some embodiments, the measurements made by the radiation sensors (140a and 140b) can be used by a user to determine if there are any problems associated with any of the EMR emitters (132a and 132b). For example, an unusually low EMR intensity emitted, may indicated that the EMR emitters (132a and 132b) may need repair and/or replacement. In this manner, having a real-time performance measurement of each EMR emitter (132a and 132b) allows the system to ensure that the object is sufficiently treated, regardless of whether one or more EMR emitters (132a and 132b) are underperforming. Some EMR emitters (and associated drivers, ballasts, bulbs, etc.) may only be 40-60% efficient, and so accounting for such inefficiencies allows the system to determine the true amount of EMR reaching the object, and therefore the true minimum duration of time to sufficiently treat the object.

The radiation sensors (140a and 140b) are configured to receive first pass radiation from the EMR emitters (132a and 132b). First pass radiation refers to radiation from the EMR emitters (132a and 132b) that reaches a destination (e.g., object, sensors, etc.) without reflecting off any surface. The radiation sensors (140a and 140b) are configured to only receive radiation from respective EMR emitters (132a and 132b). For example, the radiation sensor 140a only receives radiation from EMR emitter 132a and the radiation sensor 140b only receives radiation from EMR emitter 132b. The radiation sensors (140a and 140b) can be mounted near the base of respective EMR emitters (132a and 132b) just outside of the reactor chamber 130. In some embodiments, the radiation sensors (140a and 140b) are located equal two or less than about two inches from the respective EMR emitter (132a and 132b). The radiation sensors (140a and 140b) can be mounted on non-reflective standoffs such that the EMR sensors (140a and 140b) do not pick up radiation from EMR emitters (132a and 132b) that are not associated with the radiation sensors (140a and 140b). For example, the radiation sensor 140a can correspond to the EMR emitter 132a and the radiation sensor 140b can correspond to the EMR emitter 132b. Mounting close to the base of the EMR emitters (132a and 132b) reduces variation in the radiation sensor (140a and 140b) readings. In some embodiments, the radiation sensors (140a and 140b), if the EMR emitters (132a and 132b) are located outside of the reactor chamber 130, can still receive EMR. In some embodiments, the radiation sensors (140a and 140b) are located outside of the reactor chamber 130 in a chamber defined by the housing and receive the EMR through a transparent window. In this manner each radiation sensor (140a and 140b) can provide EMR measurements from only one respective EMR emitter (132a and 132b), thereby providing real-time accurate measurement data for each EMR emitter (132a and 132b) individually.

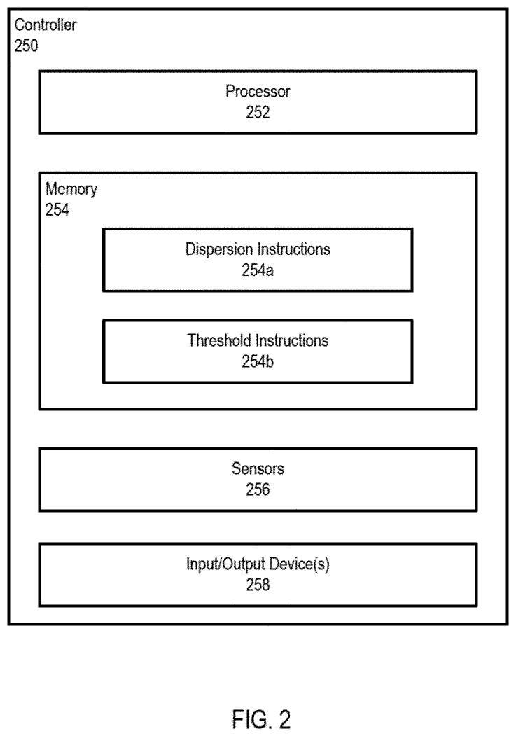

The controller 150 is configured to control and/or facilitate the operation of the EMR system 100. The controller 150 can communicatively and/or operably couple to the EMR emitters (132a and 132b), the radiation sensors (140a and 140b), and/or the access door 120. The structure of the controller is discussed further in reference to FIG. 2.

In some embodiments, the controller 150 communicatively and operatively couples to the access door 120. Based on a switch, sensors, or the like in the access door 120, the controller 150 can alter functionality of the EMR system 100. For example, if the access door 120 is in any position other than the closed position (e.g., such as slightly open to fully open), the controller 150 can stop the operation of the EMR system 100. Stopping the operation of the EMR system 100 can include shutting off the EMR emitters (132a and 132b). As another example, if the access door 120 is in a closed position, the controller 150 can lock the access door 120 from opening. As yet another example, the controller 150 can display (e.g., on a screen, indicator light, etc.) an indication of the door status (e.g., open position or closed position).

The controller 150 can control the functionality of the EMR emitters (132a and 132b). For example, the controller 150 can activate the EMR emitters (132a and 132b) for a predetermined amount of time, at a predetermined intensity, and/or in a particular pattern. The reading from radiation sensors (140a and 140b) can be used to determine the first pass radiation. Based on the orientation of the reactor chamber 130, the holders 134, the EMR emitters (132a and 132b), and the object to be treated, the controller 150 can determine first pass radiation, close second pass radiation and second pass radiation on the object. First pass radiation is the radiation that travels from the EMR emitters (132a and 132b) directly to the object being treated without reflection. The intensity of first pass radiation is related to the intensity of the EMR emitter (132a and 132b) and the distance from the EMR emitter (132a and 132b) and the object being treated. Close-second pass radiation is radiation that is reflected off a reflective surface in close proximity to the EMR emitter (132a and 132b). In some embodiments, close proximity can be defined as the radiation that reflects back off the reflective surface and radiates within the primary beam angle, where the primary beam angle corresponds to beam angle generated by the first pass radiation. Reflections outside of the primary beam angle may be second pass radiation. Close-second pass radiation is dependent on the reflectivity of the reflective surface, the distance between the reflection and the object being treated, and the transmissive properties of the EMR emitter (e.g., if the close second pass radiation passes back through the EMR emitter). In some embodiments, close proximity can less than or equal to a predefined distance. In some embodiments, close proximity can correspond to a distance corresponding to the EMR from the EMR emitter (132a and 132b) being equal to or greater than a predefined intensity. Second pass radiation is radiation that reflects off surfaces other than those in close proximity to the EMR emitter (132a and 132b). Said another way, second pass radiation is radiation that has been reflected or redirected back towards the object from any reflective surface other than the reflective surface that provides the close-second pass radiation and that forms the initial reflection beam angle. Second pass radiation can depend on the number of reflections, the reflectivity of the surfaces, and the total distance covered by the EMR.

The controller 150 can determine a target time that delivers a dose of EMR to contaminants so that the object can be treated. Continuous sub-lethal doses can result in viruses and/or bacteria developing immunities to higher EMR doses and resistance to antibiotics, so it is advantageous to treat all the surfaces of the object. In some embodiments, the target time can be the minimum time required to deliver a lethal dose to all surfaces of the object. In some embodiments, target time can be greater than the minimum time to deliver a lethal does to all surfaces of the object. For example, the target time can be about 101%, about 102%, about 103%, about 104%, about 105%, about 110%, about 115%, about 120%, about 130%, about 150%, about 200%, or more of the minimum time. As another example, the target time can be the minimum time plus an added amount of time (e.g., about 1 second, about 2 seconds, about 3 seconds, about 4 seconds, about 5 seconds, about 10 seconds, about 30 seconds, about 1 minute, about 2 minutes, about 5 minutes, etc.). In some embodiments, the controller 150 can determine the target time based on the geometry of the object and the location of the object within the reactor chamber 130. For example, the controller 150 can determine the minimum radiation intensity on the object based on the geometry of the reactor chamber 130 and the object within. In some embodiments, the controller 150 can include predetermined settings based on calibration information. For example, the predetermined settings can include a setting for if the reactor chamber is at ¼ capacity, ½ capacity, % capacity, and full capacity. In some such embodiments, the predetermined settings may correspond to user instructions and user-selectable settings or cycles to identify holder 134 and/or object position within the reactor chamber 130 so that the modeled or expected surface, face, area, point, etc. of minimum exposure on the object and/or the expected exposure per time can depend at least partially on the instructions and/or user-selectable settings or cycles. In some instances, for example, a relatively smaller object (e.g., taking up ¼ capacity) with a holder 134 closer to the bottom of the reactor chamber 130 than the top, may have a top face that is a further distance from an EMR emitter disposed towards the top of the reactor chamber 130 when compared to a larger or taller object (e.g., taking up ½ capacity, or ¼ capacity but relatively taller and narrower), which would have a top face that is relatively closer to the EMR emitter. User instructions, for example, may instruct the user to place an object in a certain location within the reactor chamber 130 (e.g., on a certain holder in instances with multiple holders, and/or within a certain location on the holder). Models may be defined accordingly.

In operation, the controller 150 can be configured to receive a start/stop signal configured to activate or deactivate the EMR system 100. In some embodiments, the controller 150 can receive a cycle setting, each cycle configured to deliver at least a minimum exposure to all surfaces of the object depending on the capacity of the reactor chamber 130. The minimum exposure corresponds to the minimum exposure needed to treat the object. In some embodiments, the controller 150 can receive an indication that a safety latch or the like of the access door 120 is closed. The controller 150 can provide an indication to the user whether the access door 120 is opened or closed. Once the controller 150 determines that the access door 120 is closed, cycle setting is selected, and the start/stop signal is set to start, the controller 150 can activate the EMR emitters 132a and 132b.

In some embodiments, the controller 150 is integrated with the housing 110. In some embodiments, the controller 150 is a device external of the housing 110 and is communicatively coupled to the other components of the EMR system 100 via a wired and/or wireless (e.g., Bluetooth, Wi-Fi, etc.). In some embodiments, the controller 150 is located on a user device (e.g., tablet, cell phone, computer, etc.) configured to communicatively couple to the EMR system 100. In some embodiments, the functionality of the controller 150 is controlled via an application on a user device.

FIG. 2 is a schematic diagram of a controller 250 (e.g., structurally and/or functionally similar to the controller 150 of FIG. 1), according to an embodiment. The controller 250 is configured to control the operations of a EMR system (e.g., functionally and/or structurally similar to the EMR system 100 of FIG. 1). The controller 250 can be a standalone system or can be integrated into the EMR system. The controller 250 includes a processor 252, a memory 254 including dispersion instructions 254a and threshold instructions 254b, sensors 256, and input/output device(s) 258. The components of the controller 250 may be integrated together on one device or may be located in various locations around the EMR system. For example, the processor 252 and the memory 254 can be located away from EMR emitting components while the sensors 256 can be located near the EMR emitting components to take measurements.

The processor 252 can be, for example, a hardware based integrated circuit (IC), or any other suitable processing device configured to run and/or execute a set of instructions or code. For example, the processor 252 can be a general-purpose processor, a central processing unit (CPU), an accelerated processing unit (APU), an application specific integrated circuit (ASIC), a field programmable gate array (FPGA), a programmable logic array (PLA), a complex programmable logic device (CPLD), a programmable logic controller (PLC) and/or the like. The processor can be operatively coupled to the memory 254 through a system bus (e.g., address bus, data bus, and/or control bus).

The memory 254 can be, for example, a random-access memory (RAM), a memory buffer, a hard drive, a read-only memory (ROM), an erasable programmable read-only memory (EPROM), and/or the like. In some instances, the memory 254 can store, for example, one or more software programs and/or code that can include instructions to cause the processor 252 to perform one or more processes, functions, and/or the like. In some implementations, the memory 254 can include extendable storage units that can be added and used incrementally. In some implementations, the memory 254 can be a portable memory (e.g., a flash drive, a portable hard disk, and/or the like) that can be operatively coupled to the processor 252. In some instances, the memory 254 can be remotely operatively coupled with a compute device (not shown). For example, a remote database device can serve as a memory and be operatively coupled to the compute device.

The dispersion instructions 254a of the memory 254 can be executed by the processor 252 to determine a dispersion pattern of EMR on an object that is being treated. In some embodiments, the dispersion instructions 254a includes instructions to use the data from radiation sensors (e.g., the radiation sensors 132a and 132b of FIG. 1). In some embodiments, the dispersion instructions 254a includes instructions for receiving information regarding the configuration of the EMR system. For example, the configuration can include the location of the EMR emitters, the type of EMR emitter, the number of EMR emitters, the intensity of the EMR emitted by the EMR emitters, shape of the reaction chamber, reflectivity of the reaction chamber walls (also referred to herein as a reflectivity factor), location of the holder(s), material of the holder(s), location of object, size of object, orientation of the object, and/or the like. The configuration of the EMR system can be inputted (e.g., by a user, by a preset, etc.). In some embodiments, at least a portion of the configuration of the EMR system can be determined. For example, sensors, such as the sensors 256, can detect the location, size, and/or configuration of the object relative to the rest of the reactor chamber. In some embodiments, the dispersion instructions 254a can includes instructions for generating a model of the reactor chamber for modeling EMR. In some embodiments, the dispersion instructions 254a include instructions for storing a pre-generated model of the reactor chamber. In some embodiments, the dispersion instructions 254a include instructions for generating a model of the object to be treated.

The dispersion instructions 254a can include instructions for the processor 252 to simulate, determine, and/or measure the dispersion of the EMR emitted by EMR emitters within the reactor chamber. In some embodiments, the dispersion instructions 254a can include instructions for the processor 252 to determine different types of dispersion. In some embodiments, the dispersion instructions 254a can include instructions for the processor 252 to determine first pass radiation, close second pass radiation, and/or second pass radiation. The first pass radiation, close second pass radiation, and/or second pass radiation correspond to different types of radiation dispersion. First pass radiation includes EMR that reaches the object to be disinfected directly from the EMR emitter without reflection. Close second pass radiation includes EMR that is emitted by the EMR emitter, reflects close to the EMR emitter (e.g., reflects on a closest surface to the EMR emitter), and reaches the object. Second pass radiation is EMR that is emitted by the EMR emitter and reflects off a surface outside of the close proximity of distance of the EMR emitter and then reaches the object. Second pass radiation reflects off a surface farther from the EMR emitter than close second pass radiation. In some embodiments, the dispersion instructions 254a can include instructions for the processor 252 to create continuous models for EMR dispersion. In some embodiments, the dispersion instructions 254a can include instructions to create discrete models (e.g., broken into rays, etc.) for EMR dispersion. In some embodiments, the dispersion instructions 254a can include instructions to determine a model for each EMR emitter separately.

To determine first pass radiation, the dispersion instructions 254a can include instructions to determine uninterrupted paths from the EMR emitters to the object to determine a first pass radiation model. The dispersion instructions 254a can include instructions to determine the distance from the EMR emitter directly to the surface of the object. In some embodiments, the dispersion instructions 254a can include instructions to determine if the first path radiation passes through a holder (e.g., the holder 134 of FIG. 1). The first pass radiation model can be added to a global model that models the entire reactor chamber or may be added to a model corresponding to an individual EMR emitter. In some implementations, the first pass radiation model (and any other models described herein), may model first pass radiation to a single surface or face of the object, and then use that model to estimate first pass radiation to remaining surfaces or faces of the object.

To determine the close-second pass radiation, the dispersion instructions 254a can include instructions to determine EMR paths from the EMR emitters to the object that first reflect off of a surface in close proximity with the EMR emitter before reaching the object to generate a close second pass radiation model. In some embodiments, close proximity can be defined as no more than about 5 inches, no more than about 4 inches, no more than about 3 inches, no more than about 2 inches, no more than about 1.5 inches, no more than about 1 inch, no more than about 0.9 inches, no more than about 0.8 inches, no more than about 0.7 inches, no more than about 0.6 inches, no more than about 0.5 inches, no more than about 0.4 inches, no more than about 0.3 inches, no more than about 0.2 inches, or no more than about 0.1 inches. In some embodiments, what is considered by the dispersion instructions 254a can include instructions to be close proximity to the EMR emitter can be directly related to the intensity of the EMR emitter. For example, a more intense EMR emitter might have a larger close proximity distance than a less intense EMR emitter. In some embodiments, the dispersion instructions 254a can include instructions to determine a first distance from the EMR emitter to the reflection surface and a second distance from the reflection surface to the object. The close-second pass radiation model can be added to the global model or may be added to a model corresponding to an individual EMR emitter.

To determine the second pass radiation, the dispersion instructions 254a can include instructions to determine EMR paths from the EMR emitters to the object that reflect off of at least one surface before reaching the object to generate a second pass radiation model. The second pass radiation reflects outside of the close proximity area near the EMR emitter discussed in reference in reference to close second pass radiation. The dispersion instructions 254a can include instructions to determine at least one distance such as a total distance from the EMR emitter to the object that the EMR travels. In some embodiments, the second pass radiation includes EMR that reflects off more than one surface. The second pass radiation model can be added to the global model or may be added to a model corresponding to an individual EMR emitter.

In some embodiments, the dispersions instructions 254a can include predetermined models. For example, the dispersion instructions 254a can include a dispersion model for all and/or each of the EMR emitters corresponding to predetermined configurations. For example, the predetermined configurations can correspond to an empty reactor chamber, the first object on a first holder, the first object on a second holder, a second object on the first holder, the second object on the second holder, and/or the like. Additionally or alternatively, in some implementations, for example, the predetermined configurations can correspond to a particular object and/or an object of a particular size range of sizes.

The threshold instructions 254b of the memory 254 can include instructions for the processor 252 to determine a radiation strategy to ensure that the entire surface area of the object being treated has received simultaneously a sufficient or lethal dose of EMR. The radiation strategies can include the amount of time EMR is directed at the object, the amount of power delivered to the EMR emitters, the number of EMR emitters activated, and the like. The threshold instructions 254b can include instructions to utilize one or more of the models generated and/or stored to determine the radiation strategy. In some embodiments, the threshold instructions 254b include instructions to receive at least one sensor measurement (e.g., from the radiation sensors 140a and 140b and/or from the sensor 256). For example, the sensor measurements can include EMR emitter intensity, surface reflectivity, temperature, humidity, power draw, number of EMR emitters, etc. In some embodiments, the threshold instructions 254b can include instructions to receive EMR emitter intensity from radiation sensors prior to every radiation strategy determination. This reduces the likelihood that inconsistencies from the EMR emitters affect the efficacy of the radiation schedule. For example, certain bulb-types used for EMR emitters can degrade over time and/or can lose efficiency. Thus, determining the EMR emitter intensity prior to and/or during determining the radiation schedule allows the radiation schedule to factor in any degradation. including degradation during operation. In some embodiments, the threshold instructions 254b include instructions to modify the radiation strategy continuously, periodically, and/or sporadically.

In some embodiments, the threshold instructions 254b include instructions to determine the intensity of the EMR on the object. For first pass radiation, the threshold instructions 254b include instruction to calculate the EMR intensity on the object based on the first pass radiation model, EMR emitter intensity, and the distance from the EMR emitter to the surface of the object. For close-second pass radiation, the threshold instructions 254b include instructions to calculate the EMR intensity on the object based on the close second pass radiation model, the reflectivity of the surface (e.g., and incorporate a reflectivity factor into a model or calculation), the first distance, the second distance, and EMR emitter intensity. For second pass radiation, the threshold instructions 254b can include instructions to calculate EMR intensity on the object based on the second pass radiation model, the reflectivity of the surfaces (e.g., and incorporate a reflectivity factor into a model or calculation), the number of reflections, and the total distance between the EMR emitter and the object. In some embodiments, the first pass radiation intensity, the second pass radiation intensity, and/or the third pass radiation intensity can be summed together to determine a collective intensity. In some embodiments, the threshold instructions 254b can include instructions to determine the collective intensity for each EMR emitter or for a subset off the EMR emitters. In some embodiments, the threshold instructions 254b can include instruction to determine a cold spot which corresponds to a point, surface, or face on the object that is receiving or expected to receive the least amount of EMR from the EMR emitters. In some embodiments, the cold spot can correspond to at least a portion of a surface of the object or to a point on the object. Ensuring that the cold spot receives a sufficient amount of EMR exposure can ensure that all remaining spots, surfaces, faces, etc. on the object also receives a sufficient amount of EMR exposure. Furthermore, the threshold instructions 254b include instructions to determine a hot spot corresponding to at least a portion of a surface of the object or to a point on the object that receives the most EMR. The hot spot can be utilized to set a maximum amount of time that corresponds to the maximum EMR exposure on the object divided by the EMR at the hot spot. In some embodiments, a maximum EMR exposure can be determined by determining the maximum available collective intensity from all the EMR emitters and subtracting the EMR projected to be absorbed (e.g., by the walls, holders, object itself, etc.).

Based on the collective intensity and/or the cold spot, the threshold instructions 254b can include instructions to determine a time period to meet a predefined radiation exposure threshold. In some embodiments, the predefined radiation exposure threshold is about 51,000 microwatt/cm². The time period corresponds to the amount of time the EMR emitters should be activated to deliver at least the predefined radiation exposure threshold to each surface of the object. The time period can be determined by determining lowest emitting EMR emitter based on at least one of the first pass radiation, close second pass radiation, and/or the second pass radiation and determining the time period required by dividing the predefined radiation exposure threshold by the radiation intensity of the lowest emitting EMR emitter. Utilizing the lowest emitting EMR emitter to determine the time period ensures that the object is treated as each point on the object will at least receive enough EMR to be treated. In some embodiments, the time period can be determined by the intensity of EMR on the cold spot and the time period can be determined by dividing the predefined radiation exposure threshold by the EMR intensity on the cold spot. In some embodiments, the time period is scaled or increases to provide a safety factor or safety buffer to ensure the predefined radiation exposure threshold is reached. For example, the time period can include the determined time period plus a safety buffer (e.g., 1 second, 2 seconds, 3 seconds, 4 seconds, 5 seconds, 10 seconds, 15 seconds, 30 seconds, 45 seconds, 1 minute, 2 minutes, 3 minutes, 5 minutes, 10 minutes, 15 minutes, 30 minutes, 45 minutes, 1 hour, etc.). As another example, the time period can be scaled by a safety factor (e.g., 1.1, 1.2, 1.3, 1.4, 1.5, 2, 3, 4, 5, 10, 15, etc.).

Although in some embodiments described herein the treatment time period is based on the lowest performing EMR emitter, in some embodiments, the emittance from each EMR emitter can be used to calculate the treatment time, rather ascribing the lowest emitting EMR emitter to others.

In some embodiments, the radiation strategy can include selectively turning certain EMR emitters on and off. For example, if the object includes a portion that is sensitive to large doses of EMR and/or a portion that is sensitive to heat, certain EMR emitters can be turned on and off selectively or automatically to prevent the object from being overexposed. In some embodiments, the threshold instructions 254b can include predetermined radiation strategies. For example, the threshold instructions 254b includes at least one predetermined radiation strategy which may include a EMR emitter intensity, an emission time, and which EMR emitters should be activated and when. For example, the predetermined radiation strategies can be based on capacity. In some embodiments, the capacity corresponds to a predetermined capacity such as a number of objects, size of object, volume of objects, etc. For example, the capacity could be 20 masks. For example, a first cycle can be for no more than a quarter capacity, a second cycle can be for no more than a half capacity, a third cycle can be for no more than three-quarters capacity, and a fourth cycle can be for a full capacity. In each cycle, the radiation strategy is configured to deliver the predefined radiation exposure threshold to the objects to be treated.

In some embodiments, the dispersion instructions 254a can include instructions described as associated with the threshold instructions 254b. In some embodiments, the threshold instructions 254b can include operations described as associated with the dispersion instructions 254a. In some embodiments, the dispersion instructions 254a and the threshold instructions 254b can be the same. In some embodiments, the memory 254 can include additional instructions.

The sensors 256 can be any additional sensors used by the controller 250 to determine the operation of the EMR system. In some embodiments, the sensors 256 include infrared sensors configured to determine the location and orientation of an object within the EMR system. The information from the infrared sensors can be utilized to determine an area of least exposure based on the location and orientation of the object and on the locations of the EMR emitters. In some embodiments, the sensors 256 include temperature sensors. The temperature sensors can determine if system temperatures exceed a predefined threshold to prevent thermal runaway. In response to the temperatures being above the predefined threshold, the controller 250 can cease operation of the EMR system.

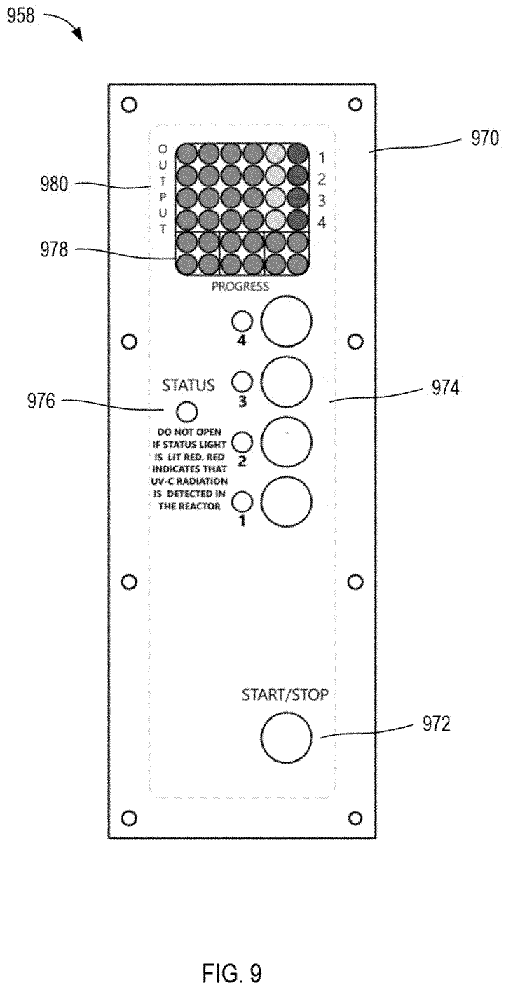

The input/output device(s) 258 can include any type of peripheral, an input device, an output device, a mouse, keyboard, microphone, touch screen, speaker, scanner, headset, printer, camera, buttons, switches, indicator lights, and/or the like. In some instances, the input/output device(s) 258 can be utilized to input operating commands into the controller 250. The input/output device(s) 258 can include any type of display, such as, for example, a Cathode Ray tube (CRT) display, a Liquid Crystal Display (LCD), a Liquid Emitting Diode (LED) display, an Organic Light Emitting Diode (OLED) display, and/or the like. The display can be used for visually displaying information (e.g., status, time left, cycle setting, etc.). In some embodiments, the input/output device(s) 258 include an interface (e.g., such as the interface shown in FIG. 9). The interface can include input buttons that can be pressed by a used to control functionality of the controller 250. The interface can also include a display and/or indicator lights that indicate information regarding the operation of the controller 250.

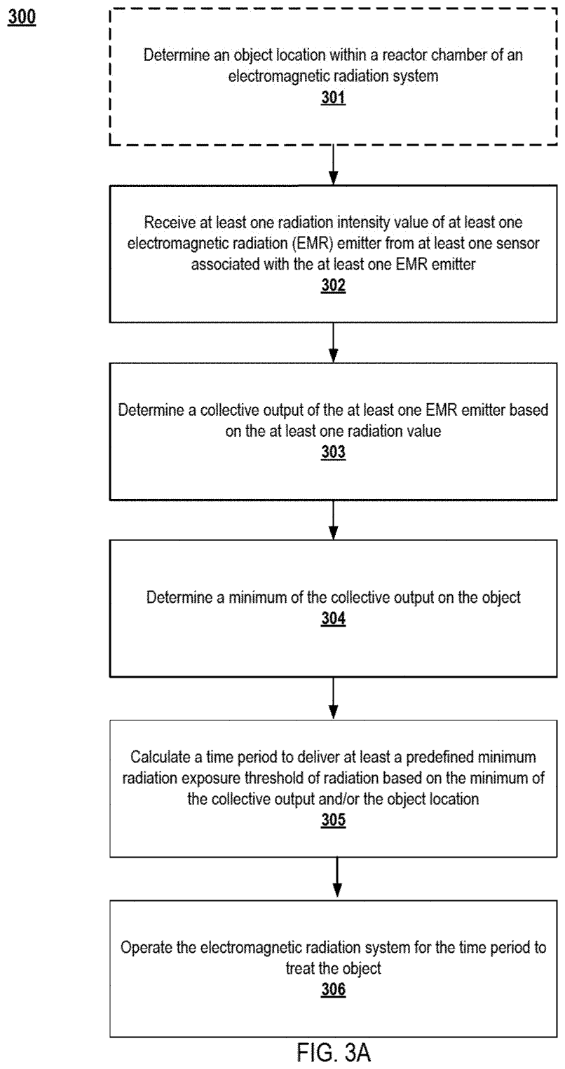

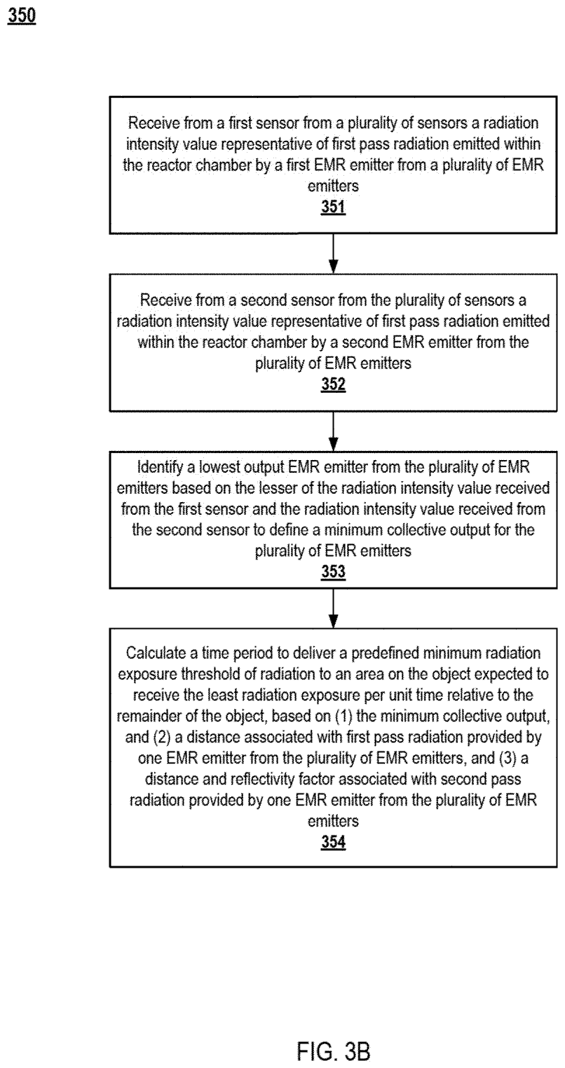

FIG. 3A is a method 300 of treating an object, according to an embodiment. The method 300 can be performed by an EMR system (e.g., functionally and/or structurally similar to the EMR system 100 of FIG. 1). The method 300 can include optionally determining an object location within a reactor chamber (e.g., functionally and/or structurally similar to the reactor chamber 130 of FIG. 1) of the EMR system at 301, receiving at least one radiation intensity value of at least one EMR emitter (e.g., functionally and/or structurally similar to the EMR emitter 132 of FIG. 1) from at least one sensor (e.g., functionally and/or structurally similar to the radiation sensors (140a and 140b) of FIG. 1) associated with the at least one EMR emitter (e.g., within or operably coupled to the reactor chamber) at 302, determining a collective output of the at least one EMR emitter based on the at least one radiation value at 303, defining a minimum of the collective output on the object at 304, calculating a time period to deliver at least a predefined minimum radiation exposure threshold of radiation based on the minimum of the collective output and/or the object location at 305, and operating an electromagnetic radiation system for the time period to treat the object at 306. In some embodiments, the method 300 can be facilitated and/or executed by a controller (e.g., functionally and/or structurally similar to the controller 150 of FIG. 1 and/or the controller 250 of FIG. 2). In some embodiments, the steps of method 300 can be stored on a memory (e.g., functionally and/or structurally similar to the memory 254 of FIG. 2) and executed by a processor (e.g., functionally and/or structurally similar to the processors 252 of FIG. 2) of the controller.

At 301, an object location is optionally determined within the reactor chamber of the EMR system. In some embodiments, the object location can be received from an information source (e.g., user, database, etc.). In some embodiments, the object location can include at least one of the object location within the reactor chamber, object size, and/or object orientation. In some embodiment, an operator can receive instructions to place the object in a particular spot based on size. In some embodiments, the object location can include additional associated information such as a maximum allowed EMR exposure. Determining the object location can include sensing the object via a sensor (e.g., infrared sensor, optical sensor, etc.) configured to measure object locations. In some embodiments, the object location can be measured by a sensors array. In some embodiments, the measurements of the sensor and/or the sensor array can be used to generate an object model. The object model can be inputted into a model of the reactor chamber. In some embodiments, 301 includes further generating the model of the reactor chamber. In some embodiments, 301 is optional.

At 302, at least one radiation intensity value is received from at least one EMR emitter from at least one sensor associated with the at least one EMR emitter. In some embodiments, the at least one sensor is located such that each sensor of the at least one sensor detects only the EMR emitted by an associated EMR emitter of the at least one EMR emitters. In some embodiments, the at least one radiation intensity value and the distance from the at least one sensor to the at least one EMR emitter are used to determine at least one origin radiation intensity, which corresponds to the radiation intensity directly emitted by a corresponding EMR emitter. In some embodiments, the at least one radiation intensity value can be determined based on the power draw by the at least one EMR emitter.

At 303, a collective output is determined based on the at least one radiation value. The collective output can include determining at least one of the first pass radiation, close second pass radiation, and/or second pass radiation. For example, the collective output can be a summation of the first pass radiation, close second pass radiation, and/or the second pass radiation. To determine the first pass radiation, close second pass radiation, and/or the second pass radiation, the model of the reactor chamber and/or the object can be used to generate a radiation dispersion model. The radiation dispersion model can be used to determine the collective output on the object. In some embodiments, the collective output can include a mapping of the object and the EMR intensity on the object. In some embodiments, the collective output can include EMR output on each surface of the object. In some embodiments, the collective output can be calculated for each of the at least one EMR emitters separately.

At 304, a minimum of the collective output on the object is determined. The minimum corresponds to lowest radiation intensity value of the collective output on the object and corresponds to the area and/or point on the object that receives the least amount of EMR during operation. In some embodiments, such as when there are multiple objects in the reactor chamber, the lowest radiation intensity value of the multiple objects is determined to be the minimum of the collective output. In some embodiments, the minimum of the collective output is determined for each of the at least one EMR emitters separately. In some embodiments, a hot pot can be determined that corresponds to a maximum of the collective output on the object.

At 305, a time period to deliver at least a predefined minimum radiation exposure threshold of radiation is calculated based on the minimum of the collective output and/or the object location. The predefined minimum radiation exposure threshold of radiation corresponds to the amount of radiation that, when delivered to a surface, is lethal to pathogens that are desired to be killed. The time period is determined by dividing the predefined minimum radiation exposure by the minimum of the collective output determined in 304. In some embodiments, the time period can be scaled by a safety factor and/or can include additional time to ensure that each surface of the object receives at least the minimum radiation exposure threshold of radiation. In some embodiments, if the hot spot multiplied by the time period will result in radiation exposure exceeding a predefined threshold, a warning notification can be generated warning that the predefined threshold may be exceeded if the EMR system is operated.

At 306, the EMR system is operated for the time period to treat the object. In some embodiments, the time period can be displayed to a user. In some embodiments, the time period can first be confirmed by a user prior to operating the EMR system. Operating the EMR system can include activating the at least one EMR emitters for the time period. In some embodiments, each or a subset of the at least one EMR emitters are activated simultaneously so that each surface of the object is treated simultaneously. In some embodiments, the time period can be altered during operation of the EMR system. For example, if the at least one sensor measures that an EMR emitter has lost intensity during operation, the time period can be increased to counteract the decrease in radiation intensity from the EMR emitter.

FIG. 3B is a method 350 of treating an object, according to an embodiment. The method 350 can be performed by an EMR system (e.g., functionally and/or structurally similar to the EMR system 100 of FIG. 1). At 351, the method includes receiving from a first sensor from a plurality of sensors a radiation intensity value representative of first pass radiation emitted within the reactor chamber by a first EMR emitter from a plurality of EMR emitters. At 352, the method includes receiving from a second sensor from the plurality of sensors a radiation intensity value representative of first pass radiation emitted within the reactor chamber by a second EMR emitter from the plurality of EMR emitters. At 353, the method includes identifying a lowest output EMR emitter from the plurality of EMR emitters based on the lesser of the radiation intensity value received from the first sensor and the radiation intensity value received from the second sensor to define a minimum collective output for the plurality of EMR emitters. At 354, the method includes calculating a time period to deliver a predefined minimum radiation exposure threshold of radiation to an area on the object expected to receive the least radiation exposure per unit time relative to the remainder of the object, based on (1) the minimum collective output, and (2) a distance associated with first pass radiation provided by one EMR emitter from the plurality of EMR emitters, and (3) a distance and reflectivity factor associated with second pass radiation provided by one EMR emitter from the plurality of EMR emitters.

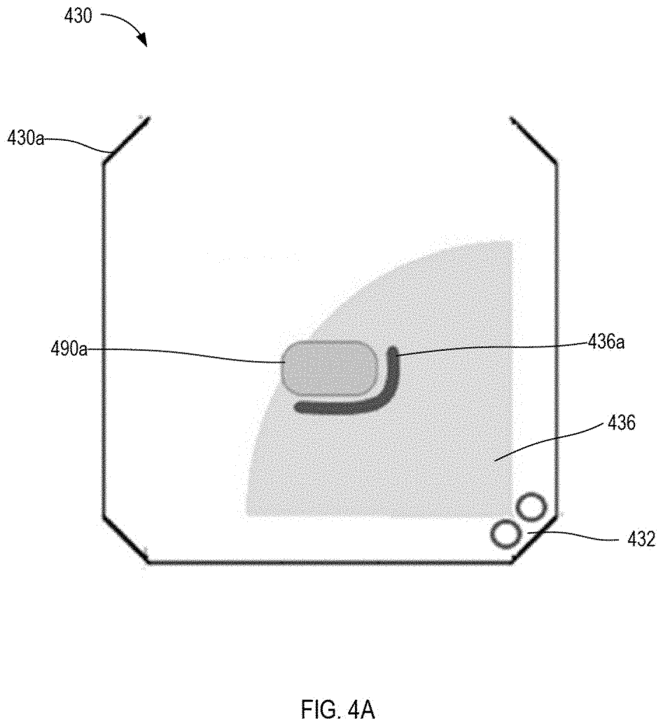

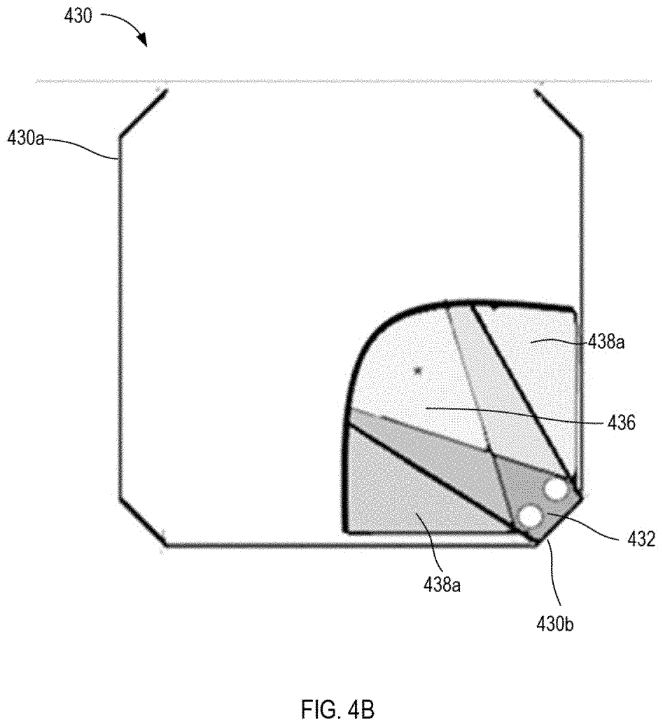

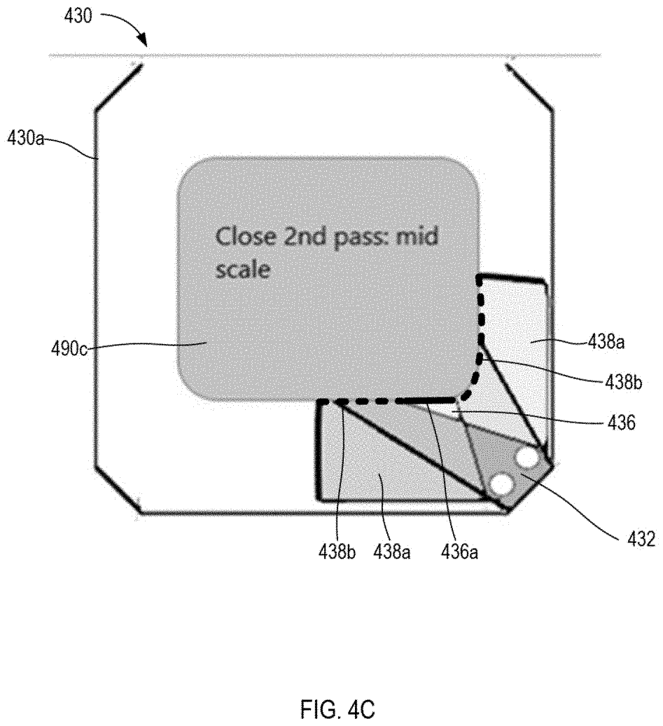

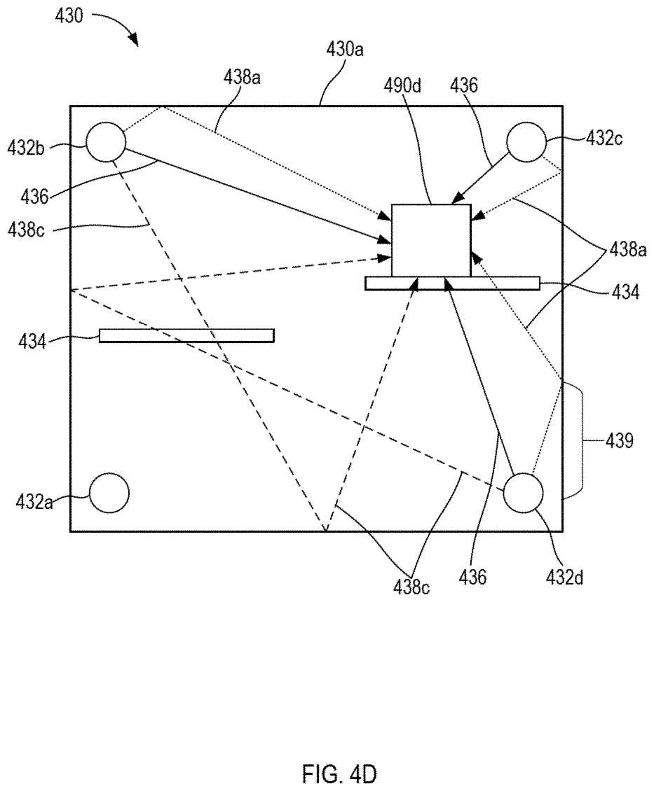

FIGS. 4A-4D depicts reactor chambers 430 (e.g., functionally and/or structurally similar to the reactor chamber 130 of FIG. 1) with EMR emitters 432 (e.g., functionally and/or structurally similar to the EMR emitters (132a and 132b) of FIG. 1). The EMR emitters 432 can be a pair of EMR emitters 432 or a single EMR emitter. FIGS. 4A-4D highlight different types of radiation dispersion patterns as well as different methods of modeling dispersion (e.g., continuously, or discretely). Only a subset of the true EMR dispersion from EMR emitters is shown in FIGS. 4A-4D for illustrative purposes and for clarity. The dispersion patterns shown in FIGS. 4A-4D can be modelled by a controller such as the controller 150 of FIG. 1 and/or the controller 250 of FIG. 2.

FIGS. 4A, 4C, and 4D depicts objects (490a, 490c, and 490d). The objects (490a, 490c, and 490d) can be modeled to generate EMR dispersion on the objects (490a, 490c, and 490d). The surfaces of the objects (490a, 490c, and 490d) can pre-modeled and input into a model. In some embodiments, the reactor chamber can include sensors that sense the objects (490a, 490c, and 490d).

FIG. 4A depicts a front view of a reactor chamber 430 with first pass dispersion 436 on an object 490a shown, according to an embodiment. The first pass dispersion 436 is EMR emitted by the EMR emitter 432 that does not reflect off a surface 430a of the reactor chamber 430. The first pass dispersion 436 is absorbed by the object at the first pass absorption area 436a. As seen in FIG. 4A, the first pass absorption area 436a only covers the portion of the object 490a that is oriented towards the EMR emitter 432 and in the direct path of the first pass dispersion 436. If it is desired for each surface of the object 490a to absorb first pass radiation, multiple EMR emitters 432 arranged around the object 490a can be oriented to provide each surface of the object 490a with first pass radiation. The intensity of the first pass radiation is dependent on the intensity of the EMR emitted from the EMR emitter 432 and the distance from the EMR emitter to the first pass absorption area.

FIG. 4B depicts a front view of a reactor chamber 430 with first pass dispersion 436 and close second pass dispersion 438a shown, according to an embodiment. The first pass dispersion 436 is similar to the first pass dispersion 436 of FIG. 4A. Additionally, FIG. 4B depicts an overlap between first pass dispersion 436 and close second pass dispersion 438a. The close second pass dispersion 438a is EMR that is emitted by the EMR emitters 432 and reflects off of the surface 430a, more specifically the near surface 430b, of the reactor chamber 430. The near surface 430b is the surface 430a that is nearest to the EMR emitters 432. The intensity of the close second pass radiation is dependent on the intensity of the EMR emitted from the EMR emitter 432, the total distance the EMR travels from the EMR emitter, and the reflectivity of the near surface 430b.

The close second pass dispersion 438a overlaps with a portion of the first pass dispersion 436. The overlapping portion includes both first pass radiation and close second pass radiation resulting in a total radiation that is a summation of the first pass radiation and the close second pass radiation. Thus, the total radiation in the overlapping portion is greater than the first pass radiation or the close second pass radiation would be at that point. FIG. 4C depicts an object 490c within the EMR dispersion which include the first pass dispersion 436a and the close second pass dispersion 438a. As seen in FIG. 4C, the object 490c includes a first pass absorption area 436a that absorbs only first pass radiation (e.g., is located only within the first pass dispersion 436) from the EMR emitter 432. The object 490c includes a first and close second pass absorption area 438b that absorbs both first pass radiation and close second pass radiation from the EMR emitter 432. In some embodiments, second pass radiation can also be modelled in addition to first pass radiation and close second pass radiation.

While FIGS. 4A-4C depict the EMR dispersion modelled as regions, EMR dispersion can be modelled as rays, as shown in FIG. 4D. FIG. 4D depicts a reactor chamber 430 with an object 490d on one of two holders 434 (e.g., functionally and/or structurally similar to the holder 134 of FIG. 1). The reactor chamber 430 includes emitters (432a, 432b, 432c, and 423d) in each corner. In the embodiment depicted in FIG. 4D, the lower left emitter 432a is deactivated. The activated EMR emitters (432b, 432c, and 432d) emit EMR radiation that include first pass radiation, close second pass radiation, and second pass radiation. FIG. 4D depicts first pass rays 436, close second pass rays 438a, and second pass rays 438c.

The first pass rays 436 are rays emitted by the EMR emitters (432b, 432c, and 432d) that directly reach the object 490d. The intensity of the first pass rays 436 depends on the intensity of the EMR emitters (432b, 432c, and 423d) and the distance from the EMR emitters (432b, 432c, and 432d) to the surface of the object 490d. In some embodiments, the first pass rays can pass through the holder 434 and the intensity of these first pass rays 436 can be dependent on how much EMR the holder 434 absorbs as well as the intensity of the EMR emitters (432b, 432c, and 432d) and the distance from the EMR emitters 432 to the surface of the object 490d.

The close second pass rays 438a are rays emitted by the EMR emitters (432b, 432c, and 432d) that reflect off the surface 430a of the reactor chamber 430 within a close distance 439. The close distance 439 may be predefined or may be dependent on the intensity of the EMR emitters (432b, 432c, and 432d). For example, if an EMR emitter 432 can a greater intensity, the close distance 439 can be greater. The intensity of the close second pass rays 438a depends on the intensity of the EMR emitters (432b, 432c, and 432d), the distance the EMR travels from the EMR emitter (432b, 432c, and 432d) to the object 490d, and the reflectivity of the surface 430a. In some embodiments, the close second pass rays 438a can pass through the holder 436 and the intensity of these close second pass rays 438a can be dependent on how much EMR the holder 434 absorbs.

The second pass rays 438c are rays emitted by the EMR emitters (432b, 432c, and 432d) that reflect off the surface 430a of the reactor chamber 430 outside of the close distance 439. The intensity of the second pass rays 438c depends on the intensity of the EMR emitters (432b, 432c, and 432d), the distance the EMR travels from the EMR emitter (432b, 432c, and 432d) to the object 490d, and the reflectivity of the surface 430a. In some embodiments, the second pass rays 438c can pass through the holder 436 and the intensity of these second pass rays 438c can be dependent on how much EMR the holder 434 absorbs.

The EMR dispersion depicts in FIGS. 4A-4D can be utilized in determining a minimum exposure on the object (e.g., cold point). The cold point can correspond to a point on a surface with a minimum EMR exposure based on the modeled EMR dispersion. In some embodiments, the configuration of the objects (490a, 490c, and 490d) can affect which surface or point is determined to be the cold point. For example, placing an object above the midpoint of an upper set of EMR emitters (e.g., the EMR emitters 432b and 432c of FIG. 4D) would have a cold point at the top (e.g., top surface, top face, etc.) of the object. The different types of dispersion can be summed together to determine the cold point. As an example, the object 490d would have a cold spot on the left side as the EMR emitter 432a is deactivated and thus causes the left side to absorb the least EMR. In some embodiment, a portion of an object can receive no first pass radiation because of self-obstruction by the object. The cold spot can correspond to the spot that receives no first pass radiation. In some embodiments, the cold spot can correspond to the point on the object that receives first pass radiation but at a furthest distance from the EMR emitter (e.g., the least amount of first pass radiation). In some embodiments, summation of the first pass radiation, close second pass radiation, and second pass radiation can include reduction modifiers, which can factor in reflections, and/or beam angle overlap.

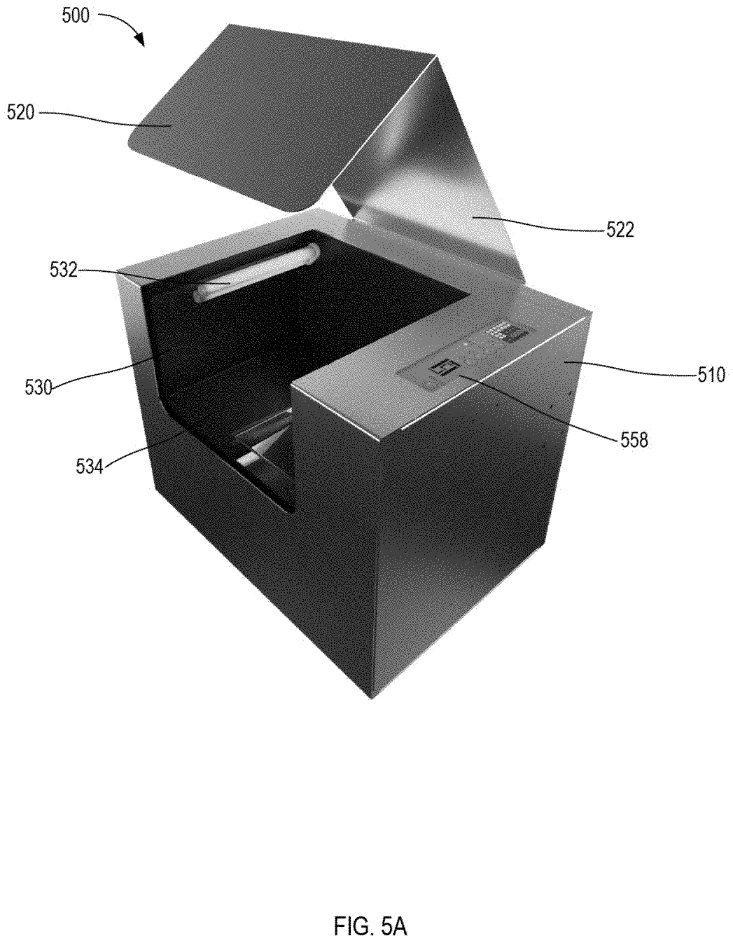

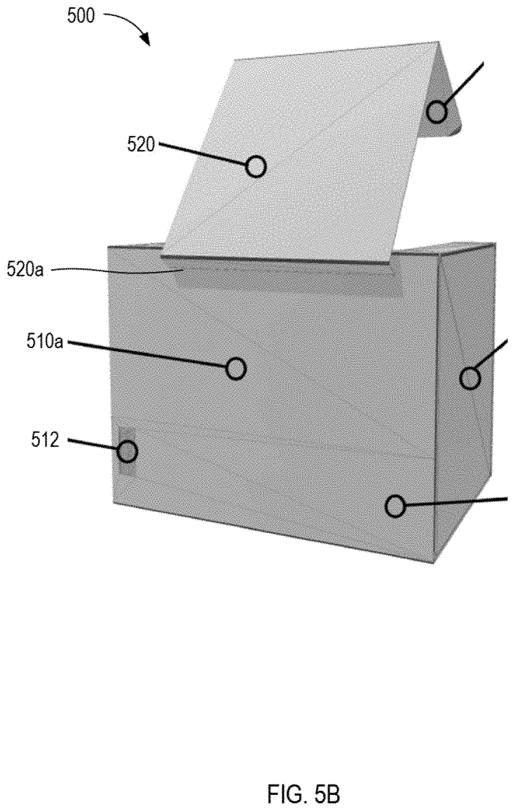

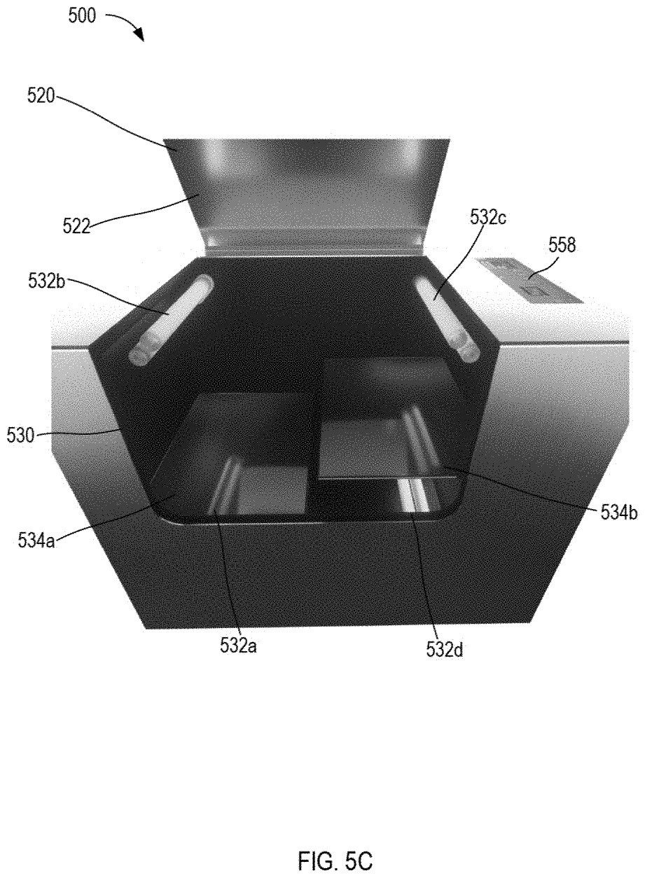

Referring generally to FIGS. 5A-5C, an EMR system 500 (e.g., structurally and/or functionally similar to the EMR system 100 of FIG. 1) is shown. The EMR system 500 can be utilized to treat objects. The EMR system 500 includes a housing 510 (e.g., functionally and/or structurally similar to the housing 110 of FIG. 1), an access door 120 (e.g., functionally and/or structurally similar to the access door 120 of FIG. 1) including a reflective surface 522, a reactor chamber 530 (e.g., functionally and/or structurally similar to the reactor chamber 130 of FIG. 1) defined within the housing 510, holders 534 (e.g., functionally and/or structurally similar to the holder 134 of FIG. 1), EMR emitters 532 (e.g., functionally and/or structurally similar to the EMR emitters (132a and 132b) of FIG. 1), and an input/output device 558 (e.g., functionally and/or structurally similar to the input/output device(s) 258 of FIG. 2). The embodiments depicted in FIGS. 5A-5C show an exemplary EMR system, however, other configurations of EMR systems are possible.

FIG. 5A depicts a perspective view of a front of an EMR system 500, according to an embodiment. The housing 510 of the EMR system 500 is a rectangular prismatic structure formed of a metal. The housing 510 provides structure of the EMR system 500. The housing 510 defines the reactor chamber 530 which can be selectively closed by the access door 520. The access door 520 is a hinged door that is operably coupled to the housing 510 so that it can operate between an open position (e.g., as depicted in FIG. 5A) and a closed position (e.g., preventing access to the reactor chamber 530 and preventing EMR from escaping the reactor chamber 530). The housing 510 is open as to provide top and front access to the reactor chamber 530. The access door 520 is a corresponding shape so that it covers the openings into the reactor chamber 530 when the access door 520 is in a closed position. The inside (e.g., relative to the housing 510) surface of the access door 520 is a reflective surface 522. The reflective surface 522 is configured to reflect EMR so that the reflective surface 522 can form the walls of the reactor chamber 530, when the access door 520 is in the closed position.

The reactor chamber 530 has a roughly rectangular prism shape within the housing 510. In some embodiments, the reactor chamber 530 can have an alternate shape (e.g., cylindrical, spherical, triangular, etc.). The walls of the reactor chamber 530 are reflective so that they can reflect EMR within the reactor chamber 530. The reactor chamber 530 can have curved edges and/or angled edges to better reflect EMR towards the center of the reactor chamber 530.

The reactor chamber 530 houses the holder 534 and the EMR emitters 532. The EMR emitters 532 are mounted to the housing 510 and extend into the reactor chamber 530. The EMR emitters 532 are located along the periphery of the reactor chamber 530 to provide EMR to objects that may be located within the reactor chamber 530 on the holder 534. The holder 534, in FIG. 5A, is a transparent surface on which an object (not shown) can be placed. In some embodiments, the holder 534 can be located in a different portion of the reactor chamber 530. In some embodiments, the holder 534 can be a hook, pin, loop, or other feature that can support and/or suspend an object.

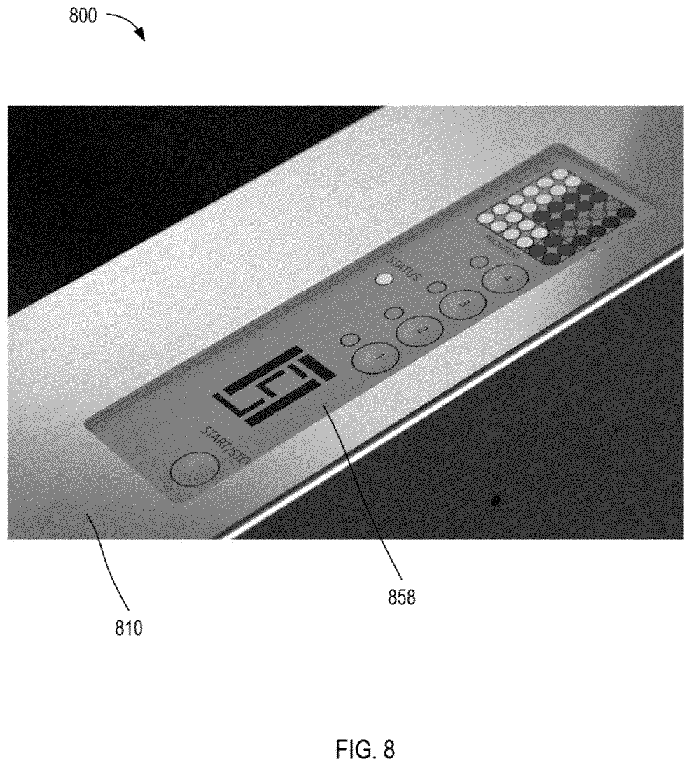

The input/output device 558 is located on the top surface of the housing 510. The input/output device 558 is configured to receive inputs from an operator and to display information to the operator. For example, the input/output device 558 can receive operating inputs from the operator, the operating inputs corresponding to the type of object inputted, the desired type of treatment cycle, a start command, and/or the like. As another example, the input/output device 558 can display EMR system 500 status, warnings, maintenance notifications, cycle selected, cycle time remaining, and/or the like. In some embodiments, the input/output device 558 can be operably coupled to a controller (e.g., functionally and/or structurally similar to the controller 150 of FIG. 1 and/or the controller 250 of FIG. 2), which can facilitate the functionality of the EMR system 500. The input/output device 558 is discussed in further detail in reference to FIG. 8.

FIG. 5B depicts a perspective view of the back of the EMR system of FIG. 5A. The access door 520 is mounted to the back housing 510a via a hinge 520a. In some embodiments, hinge 520a can include features (e.g., locks, pistons, etc.) that allow for the access door 520 to maintain an open position. This allows for an operator to release the access door 520 in the open position while loading the reactor chamber. In some embodiments, the hinge 520a can include a pin, switch, or other feature that an operator can actuate to lock the access door 520 in an open or a closed position.

The back housing 510a includes an electrical port 512. The electrical port 512 is configured to couple the EMR system to an external power source. For example, the electrical port 512 can include connectors for electrically coupling the EMR system to a generator, grid power, outlet, transformer, and/or the like. In some embodiments, the electrical port 512 can include multiple types of connectors. For example, the electrical port 512 can include a 110V connector for operating certain portions of the EMR system and a 220V connector for operating other portions of the EMR system.

FIG. 5C depicts a front view of the EMR system 500 of FIG. 5A. The EMR emitters (532a, 532b, 532c, and 532d) are arranged in a top configuration (532b and 532c) and a bottom configuration (532a and 532d). The holders (534a and 534b) are arranged in an offset position with the holder 534a being oriented lower than the holder 534b. The offset position allows for objects of varying sizes and shapes to be placed for treatment and can allow for multiple objects to be placed and treated. In some embodiments, the orientation of the holders (534a and 534b) can be reoriented based on the type of object.

In some embodiments, the reaction chamber 530 can define a maximum height corresponding to the maximum height a holder (534a and 534b) can be located and a minimum height corresponding to the minimum height a holder (534a and 534b) can be located. Establishing a minimum and maximum height can reduce the likelihood that the object is oriented in an area of the reactor chamber that does not receive sufficient EMR for treatment. For example, if an object is located against a wall of the reactor chamber, the surface(s) of the object against the wall may not receive sufficient EMR for treatment.

In some embodiments, the location of the EMR emitters (532a, 532b, 532c, and 532d) can be reconfigured so that at least one of the EMR emitters (532a, 532b, 532c, and 532d) is located in a different portion of the reactor chamber. In some embodiments, additional or fewer EMR emitters can be included in the EMR system 500.

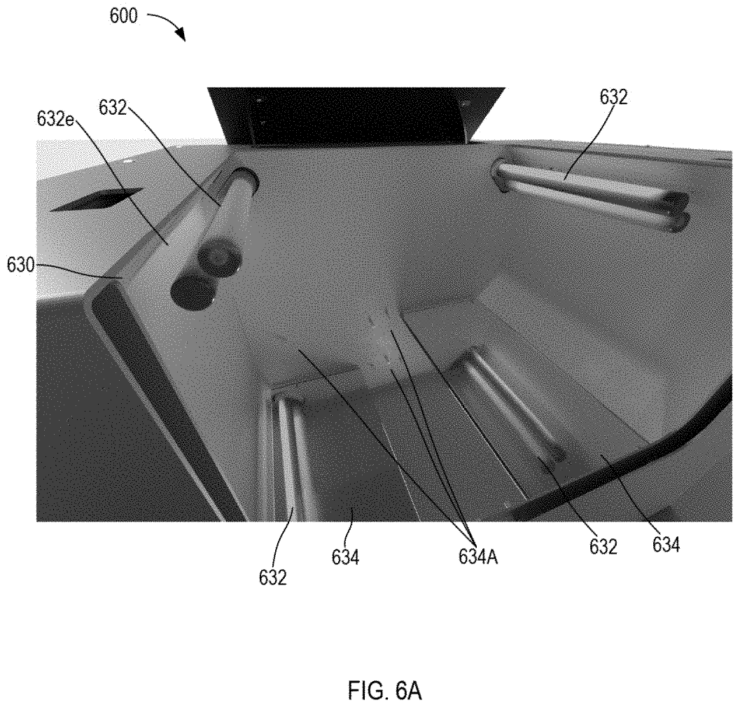

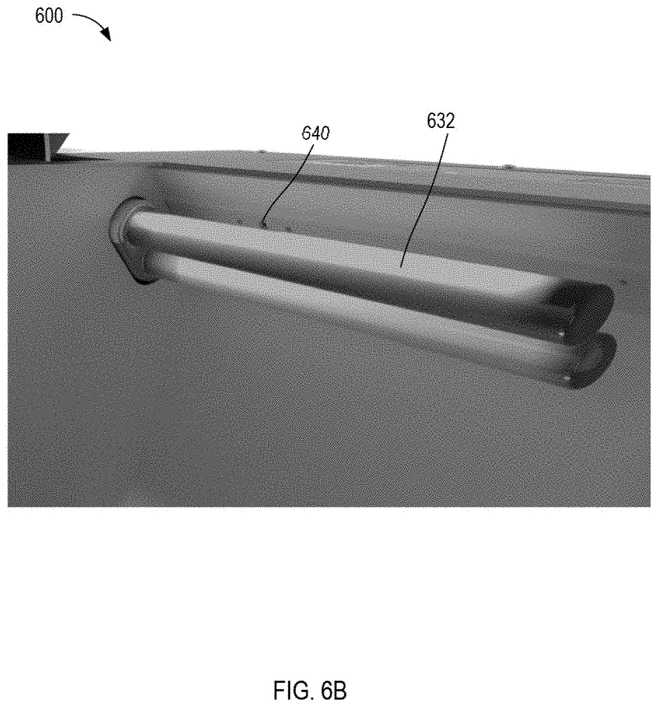

FIG. 6A depicts a front view of a reactor chamber 630 (e.g., functionally and/or structurally similar to the reactor chamber 130 of FIG. 1 and/or the reactor chamber 530 of FIGS. 5A and 5C) of an EMR system 600 (e.g., functionally and/or structurally similar to the EMR system 100 of FIG. 1 and/or the EMR system 500 of FIGS. 5A-5C), according to an embodiment. As seen in FIG. 6A, the reactor chamber 630 houses EMR emitters 632 and holders 634. The reactor chamber 630 includes additional mounting point 634a that can be utilized to reconfigure the positions of the holders 634. The mounting points 634a can include adhesives, pins, supports, and/or the like. In some embodiments, the mounting point 634a can include one or more sensors that detects the presence of an object on at least one of the holders 634. FIG. 6A also depicts a space 632e between the EMR emitters 632 and walls of the reactor chamber 630. The space 632e allows for the EMR emitted by the EMR emitters 632 to bounce off the wall directly behind the EMR emitter 632 and emit close second pass radiation. FIG. 6B shows an example implementation in which a wall of the reactor chamber 630 defines an opening through which the radiation sensor 640 is disposed, such that the sensor 640 is spaced or offset from within the reactor chamber 630 so that the only radiation it receives is first pass radiation from EMR emitter 632, thereby providing an accurate and precise performance measurement of EMR emitter 632 in real-time.

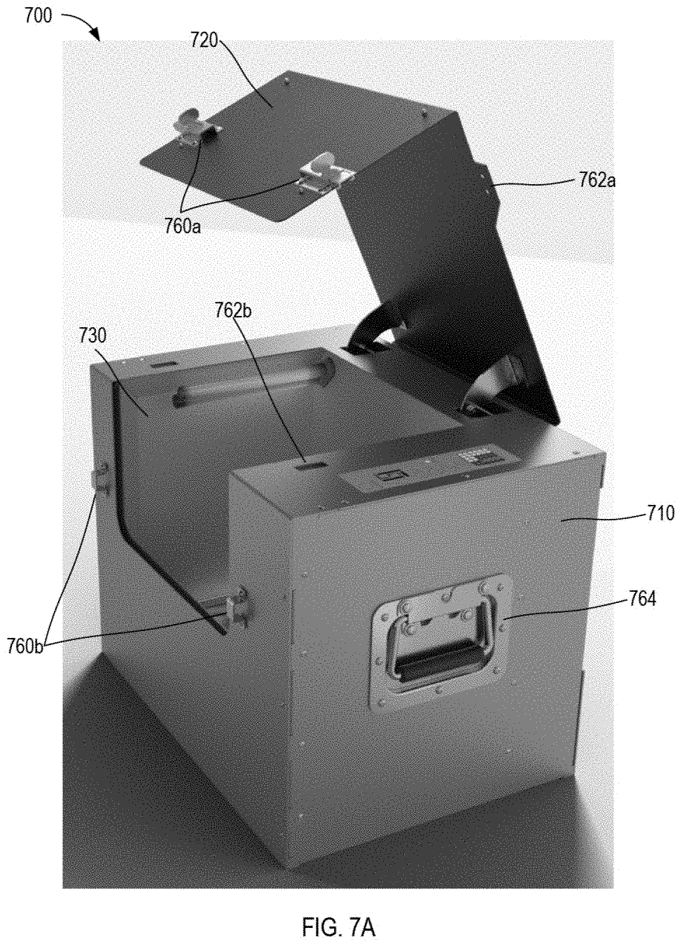

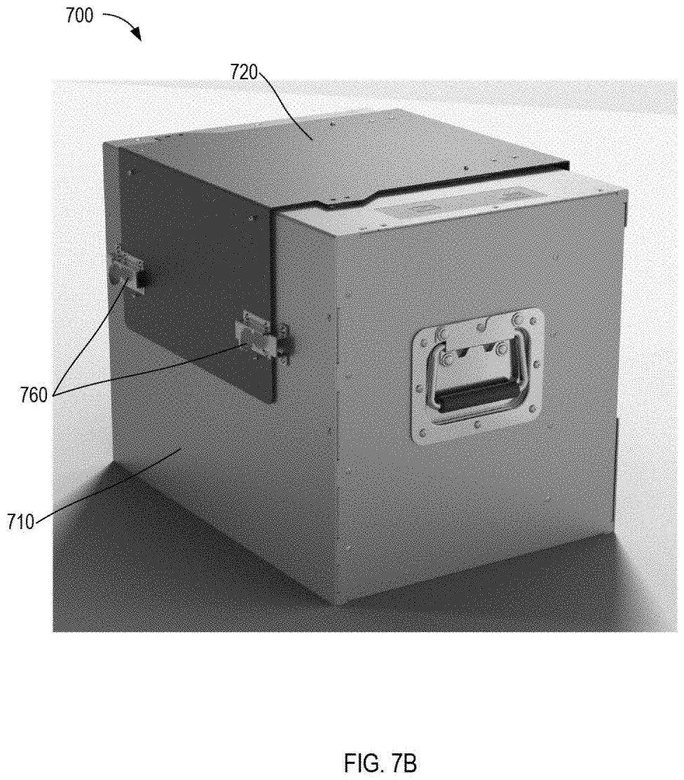

FIG. 7A depicts a perspective view of an EMR system 700 (e.g., functionally and/or structurally similar to the EMR system 100 of FIG. 1) with additional hardware in an open state, according to an embodiment. The EMR system 700 includes additional hardware on an access door 720 (e.g., functionally and/or structurally similar to the access door 120 of FIG. 1) including brackets 760a. The fasteners 760a correspond to receiving fasteners 760b on a housing 710 (e.g., functionally and/or structurally similar to the housing 110 of FIG. 1). When the access door 720 is in a closed state, the fasteners 760a can be engaged with the receiving fasteners 760b to lock the access door 720 in a closed position as seen in FIG. 7B. In some embodiments, the EMR system 700 can only operate when the access door 720 is locked. The access door 720 further includes a lock tab 762a which corresponds to a lock slot 762b on the housing. When the access door 720 is in the closed position, the lock tab 762a aligns with the lock slot 762b so that a lock (e.g., padlock, combination locket, etc.) can be fed through the lock tab 762a and the lock slot 762b to lock the access door 720 in a closed position.

The housing 710 includes handle hardware 764. The handle hardware 764 allows for the EMR system 700 to be carried when not in use. This provides the EMR system 700 with repositionability so that it can be used in multiple settings. In some embodiments, the housing 710 can include multiple handle hardware 764 in different positions to allow for multiple carrying configurations and/or to provide handle hardware 74 for multiple operators to carry the EMR system 700.

FIG. 8 depicts an input/output device 858 (e.g., functionally and/or structurally similar to the input/output device 258 of FIG. 2 and/or the input/output device 585 of FIG. 5A) of an EMR system 800 (e.g., functionally and/or structurally similar to the EMR system of FIG. 1 and/or the EMR system 500 of FIGS. 5A-5C), according to an embodiment. The input/output device 858 is integrated with the housing 810 (e.g., functionally and/or structurally similar to the housing 110 of FIG. 1). In some embodiments, the input/output device 858 can be a remote device (e.g., wireless device, cloud device, etc.). In some embodiments, the input/output device 858 can be a device connected to the EMR system 800 via a wired connection. The input/output device 858 is configured to display and receive information regarding the operation of the EMR system 810. The layout and function of the input/output device 858 is described further in reference to FIG. 9.

FIG. 9 depicts a top view of an input/output device 958 (e.g., functionally and/or structurally similar to the input/output device 858 of FIG. 8), according to an embodiment. The input/output device 958 includes a faceplate 970. The faceplate 970 provides the input/output device 958 with structure and houses the rests of the features of the input/output device 958. On the faceplate 970 is a start/stop button 972, cycle selection buttons 974, a status light 976, a progress bar 978, and an output bank 980. In some embodiments, different configurations of input/output device 958 can be used.

The cycle selection buttons 974 are buttons corresponding to different predetermined cycles of the EMR system. For example, a first button can correspond to a cycle for a quarter-full reactor chamber, a second button can correspond to a cycle for a half-full reactor chamber, a third button can correspond to a cycle for a three-quarters-full reactor chamber, and a fourth button can correspond to a full reactor chamber.

The start/stop button 972 is used to start and/or stop the operation of the EMR system (e.g., functionally and/or structurally similar to the EMR system 100 of FIG. 1). In some embodiments, the start/stop button 972 can only function if the access door (e.g., functionally and/or structurally similar to the access door 120 of FIG. 1) is in a closed position to prevent EMR from escaping out of the EMR system and to an operator. Once activated, the status light 976 can indicate if EMR is detected within the housing and/or the reactor chamber. The progress bar 978 can indicate the progress of the cycle. For example, it can indicate what portion of the cycle has been completed/is left or it can indicate what phase of the cycle is occurring. As another example, the progress bar can include 30% completion, 60% completion, and 90% completion, where completion corresponds to desired EMR delivery.

The output bank 980 includes rows of output lights that can indicate the status of the EMR emitters of the EMR system. Each row corresponds to one of the EMR emitters. For example, a first row can correspond to a first EMR emitter, a second row can correspond to a second EMR emitter, a third row can correspond to a third EMR emitter, and a fourth row can correspond to a fourth EMR emitter. The color of the output lights indicates the status of the respective EMR emitter. For example, a green light can indicate the EMR emitter is operating within a nominal output range, a yellow light can indicate the EMR emitter is operating below a nominal output range, and a red light can indicate the EMR emitter has no output detected.

FIGS. 10A-10C depict a configuration of delivering EMR to an object using a single EMR emitter 1032 (e.g., functionally and/or structurally similar to the EMR emitter (132a and 132b) of FIG. 1). The configurations shown in FIGS. 10A-10C can include and/or be a part of an EMR system, such as the EMR system 100 of FIG. 1.

FIG. 10A depicts a side view of the single EMR emitter 1032 configuration, according to an embodiment. The single EMR emitter 1032 configuration is configured to deliver EMR to an object 1090 from a single source EMR emitter 1032. This configuration routes the EMR to the object 1090 such that all surfaces of the object 1090 receive EMR simultaneously. The object 1090 can be located on a holder 1034 (e.g., functionally and/or structurally similar to the holder 134 of FIG. 1). The EMR emitter 1032 is optically coupled to a set of fiber optic conduits 1092. The fiber optic conduits 1092 direct EMR from the EMR emitter 1032 to a set of diffusers 1094 with diffuse the EMR into diffused EMR 1032e. FIG. 10B depicts how the diffusers 1094 diffuse the EMR out radially into diffused EMR 1032e from the diffuser 1094. The diffused EMR 1032 reaches some surfaces of the object 1090 and also reflect off reflective portion 1022. The reflective portions 1022 reflect the EMR onto the surfaces of the object 1090 so that the object 1090 can have all surfaces treated simultaneously. In some embodiments, such as the embodiment depicted in FIG. 10C, the single EMR emitter 1032 configuration can include additional reflective portions 1022.

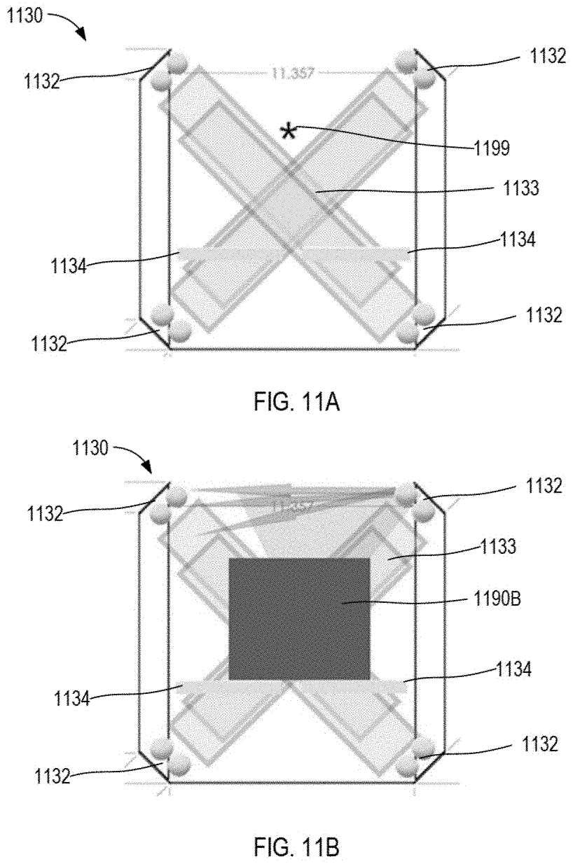

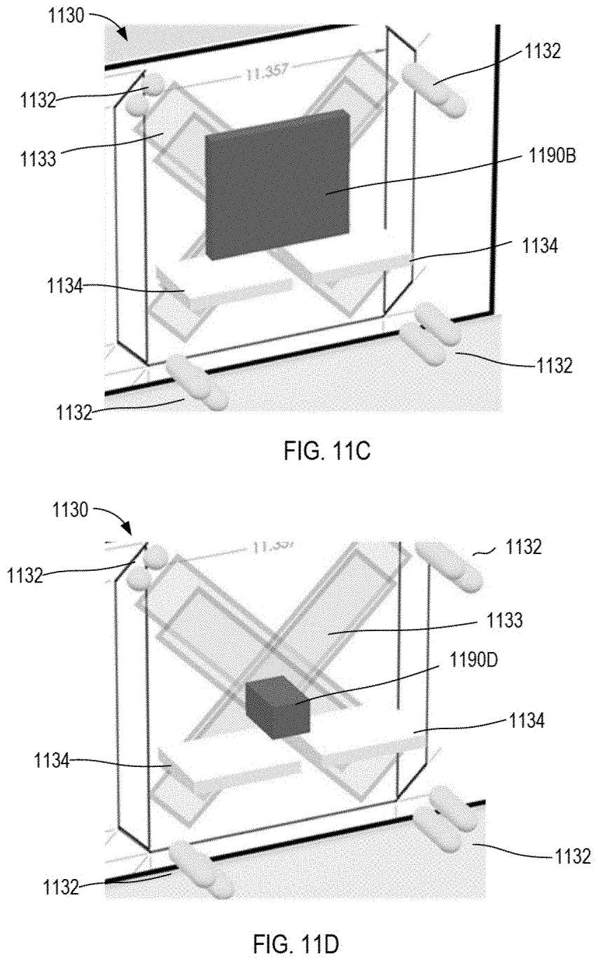

FIG. 11A depicts a front view of a reactor chamber 1130 (e.g., functionally and/or structurally similar to the reactor chamber 130 of FIG. 1) with dispersion beams 1133 shown, according to an embodiment. The reactor chamber 1130 can include or be a portion of an EMR system (e.g., functionally and/or structurally similar to EMR system 100 of FIG. 1). The dispersion beams 1133 are the EMR emitted from EMR emitters 1132 (e.g., functionally and/or structurally similar to the EMR emitters 132a and 132b of FIG. 1) and can be the highest intensity portions of the dispersion arc. The dispersion beams 1133 overlap around the centroid of the reactor chamber 1130. Outside of the dispersion beams 1133, the EMR intensity can be lower. The cold spot 1199 corresponds to the lowest EMR intensity point within the reactor chamber 1130. Portions of the dispersion beams 1133 pass through holders 1134 (e.g., functionally and/or structurally similar to the holders 134 of FIG. 1). In some implementations, a portion of the beam angle is composed of a section that is first pass with about a 180-degree beam angle, and close second pass with about a 180-degree beam angle. This is the highest intensity portion of the arc and it is relatively focused compared to other portions of the arc. Such high intensity tight beam angles will intersect at different distances vertically and horizontally in embodiments in which the reactor chamber is asymmetrical (e.g., slightly wider than tall), and/or angled deflector surfaces (e.g., 45 degree vertically opposed defector surfaces at both forward limits). Without obstruction, the cold spot 1199 should receive the least amount of EMR, e.g., when the system starts-up and until the average energy within the reactor chamber approaches the average energy of the 180-degree collective first and closest second pass beam within the arc.

FIG. 11B depicts the reactor chamber of FIG. 11A with a large object 1190B within. As shown, in this arrangement, the large object/obstruction 1190B has a known maximum because it is limited by the position of the holder 1134. The portions of the object 1190B that are within the beam dispersion 1133 receive the largest portion of the EMR dispersion from the EMR emitters 1132 (e.g., highest intensity where 180-degree beam intersects). The portions of the object 1190B outside of the dispersion beams 1133 such as on the top surface and/or the left and right surfaces receives relatively lower intensity first pass and/or close second pass of the beam angle, and relatively more second pass radiation. Further, more first pass radiation is absorbed at closer distances and does not become second pass, when compared to smaller objects, and more close second pass radiation is absorbed on treatment surfaces at closer distances and correspondingly does not become second pass radiation. Further, second pass that would otherwise travel further rand be further disbursed has a greater chance of being absorbed at nearer distances, with such an object 1190B. Further, surface areas that are furthest away or in the vicinity of overlapping high intensity portions of the art depend more on second pass radiation, which becomes less available due to the absorption of first pass radiation, close second pass, and/or second pass. The dispersion on the large object 1190B can be seen in perspective view in FIG. 11C. FIG. 11D depicts a perspective view of the reactor chamber 1130 of FIG. 11A with a small object 1190D within. The smaller object 1190c is located within the beam dispersion 1133. The cold spot on the small object 1190D can be the located where the EMR passes through the holder 1134. In some embodiments, the cold spot on the small object 1190D can correspond to a point that is farthest from the EMR emitters 1132. In some implementations, the back and/or front surfaces of the reactor chamber are angled (e.g., at about 45 degrees) such that EMR that is not absorbed prior to reflection from the back of the reactor chamber absorbs on its path back to the front or is redirected by such angled surfaces toward the centroid.

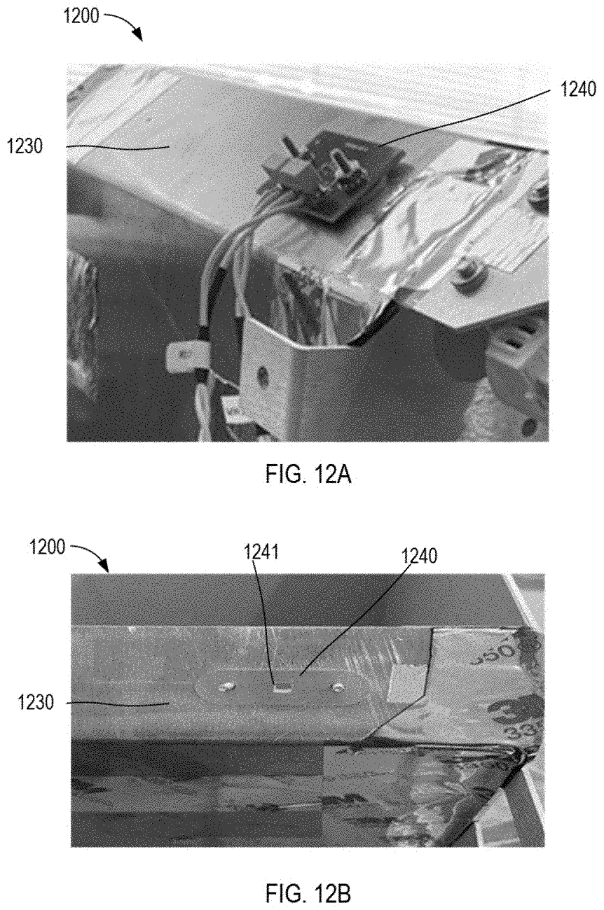

FIG. 12A depicts a sensor 1240 (e.g., functionally and/or structurally similar to the radiation sensors (140a and 140b) of FIG. 1) mounted on the outside of reactor chamber 1230 (e.g., functionally and/or structurally similar to the reactor chamber 130 of FIG. 1) of an EMR system 1200 (e.g., functionally and/or structurally similar to the EMR system 100 of FIG. 1), according to an embodiment. The sensor 1240 can be mounted to the reactor chamber 1230 so that the body of the sensor 1240 does not extend into the reactor chamber 1230 while sensing the EMR intensity of an EMR emitter. As seen in FIG. 12B, the sensor 1240 is roughly flush against the internal surface of the reactor chamber 1230. The sensor 1240 include sensor window 1241 that receives and detects EMR intensity. The sensors 1240 can be included on multiple locations of the reactor chamber 1230 to detect EMR throughout the EMR system 1200.

It should be understood that the disclosed embodiments are not intended to be exhaustive, and functional, logical, operational, organizational, structural and/or topological modifications may be made without departing from the scope of the disclosure. As such, all examples and/or embodiments are deemed to be non-limiting throughout this disclosure.

All definitions, as defined and used herein, should be understood to control over dictionary definitions, definitions in documents incorporated by reference, and/or ordinary meanings of the defined terms.

Examples of computer code include, but are not limited to, micro-code or micro-instructions, machine instructions, such as produced by a compiler, code used to produce a web service, and files containing higher-level instructions that are executed by a computer using an interpreter. For example, embodiments can be implemented using Python, Java, JavaScript, C++, and/or other programming languages and development tools. Additional examples of computer code include, but are not limited to, control signals, encrypted code, and compressed code.

The drawings primarily are for illustrative purposes and are not intended to limit the scope of the subject matter described herein. The drawings are not necessarily to scale; in some instances, various aspects of the subject matter disclosed herein can be shown exaggerated or enlarged in the drawings to facilitate an understanding of different features. In the drawings, like reference characters generally refer to like features (e.g., functionally similar and/or structurally similar elements).

The acts performed as part of a disclosed method(s) can be ordered in any suitable way. Accordingly, embodiments can be constructed in which processes or steps are executed in an order different than illustrated, which can include performing some steps or processes simultaneously, even though shown as sequential acts in illustrative embodiments. Put differently, it is to be understood that such features may not necessarily be limited to a particular order of execution, but rather, any number of threads, processes, services, servers, and/or the like that may execute serially, asynchronously, concurrently, in parallel, simultaneously, synchronously, and/or the like in a manner consistent with the disclosure. As such, some of these features may be mutually contradictory, in that they cannot be simultaneously present in a single embodiment. Similarly, some features are applicable to one aspect of the innovations, and inapplicable to others.

Where a range of values is provided, it is understood that each intervening value, to the tenth of the unit of the lower limit unless the context clearly dictates otherwise, between the upper and lower limit of that range and any other stated or intervening value in that stated range is encompassed within the disclosure. That the upper and lower limits of these smaller ranges can independently be included in the smaller ranges is also encompassed within the disclosure, subject to any specifically excluded limit in the stated range. Where the stated range includes one or both of the limits, ranges excluding either or both of those included limits are also included in the disclosure.

The phrase “and/or,” as used herein in the specification and in the embodiments, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements can optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the embodiments, “or” should be understood to have the same meaning as “and/or” as defined above. For example, when separating items in a list, “or” or “and/or” shall be interpreted as being inclusive, i.e., the inclusion of at least one, but also including more than one of a number or list of elements, and, optionally, additional unlisted items. Only terms clearly indicated to the contrary, such as “only one of” or “exactly one of,” or, when used in the embodiments, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the embodiments, shall have its ordinary meaning as used in the field of patent law.

As used herein in the specification and in the embodiments, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements can optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the embodiments, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Some embodiments described herein relate to a computer storage product with a non-transitory computer-readable medium (also can be referred to as a non-transitory processor-readable medium) having instructions or computer code thereon for performing various computer-implemented operations. The computer-readable medium (or processor-readable medium) is non-transitory in the sense that it does not include transitory propagating signals per se (e.g., a propagating electromagnetic wave carrying information on a transmission medium such as space or a cable). The media and computer code (also can be referred to as code) can be those designed and constructed for the specific purpose or purposes. Examples of non-transitory computer-readable media include, but are not limited to, magnetic storage media such as hard disks, floppy disks, and magnetic tape; optical storage media such as Compact Disc/Digital Video Discs (CD/DVDs), Compact Disc-Read Only Memories (CD-ROMs), and holographic devices; magneto-optical storage media such as optical disks; carrier wave signal processing modules; and hardware devices that are specially configured to store and execute program code, such as Application-Specific Integrated Circuits (ASICs), Programmable Logic Devices (PLDs), Read-Only Memory (ROM) and Random-Access Memory (RAM) devices. Other embodiments described herein relate to a computer program product, which can include, for example, the instructions and/or computer code discussed herein.

Some embodiments and/or methods described herein can be performed by software (executed on hardware), hardware, or a combination thereof. Hardware modules may include, for example, a processor, a field programmable gate array (FPGA), and/or an application specific integrated circuit (ASIC). Software modules (executed on hardware) can include instructions stored in a memory that is operably coupled to a processor and can be expressed in a variety of software languages (e.g., computer code), including C, C++, Java™, Ruby, Visual Basic™, and/or other object-oriented, procedural, or other programming language and development tools. Examples of computer code include, but are not limited to, micro-code or micro-instructions, machine instructions, such as produced by a compiler, code used to produce a web service, and files containing higher-level instructions that are executed by a computer using an interpreter. For example, embodiments may be implemented using imperative programming languages (e.g., C, Fortran, etc.), functional programming languages (Haskell, Erlang, etc.), logical programming languages (e.g., Prolog), object-oriented programming languages (e.g., Java, C++, etc.) or other suitable programming languages and/or development tools. Additional examples of computer code include, but are not limited to, control signals, encrypted code, and compressed code.

We claim:

1. An apparatus, comprising:
a housing defining a reactor chamber and an access door configured to transition between an open configuration to allow access to reactor chamber from outside the reactor chamber to a closed configuration to prevent access to the reactor chamber from outside the reactor chamber;
a holder configured to support within the reactor chamber an object to be disinfected;
a plurality of electromagnetic radiation (EMR) emitters disposed within the reactor chamber and configured to emit electromagnetic radiation to treat the object, the plurality of EMR emitters including a first EMR emitter and a second EMR emitter;
a plurality of sensors disposed within the housing and configured to detect radiation intensity, the plurality of sensors including a first sensor configured to detect first pass radiation from the first emitter and not from the second emitter to measure radiation intensity emitted by the first EMR emitter, and a second sensor configured to measure first pass radiation from the second emitter and not from the first emitter to identify radiation intensity emitted by the second EMR emitter; and
a controller configured to calculate a time period to meet a predefined radiation exposure threshold for a treatment cycle based on the lower of the first pass radiation detected by the first sensor and the first pass radiation measured by the second sensor, the controller configured to send a signal to identify completion of the treatment cycle in response to completion of the time period.

2. The apparatus of claim 1, wherein the reactor chamber is defined by a plurality of interior surfaces within the housing, the interior surfaces being configured such that at least 50% of EMR emitter output is absorbed the object.

3. The apparatus of claim 1, wherein interior surfaces of the reactor chamber are lined with at least one of aluminum sputtered glass and polytetrafluoroethylene (PTFE).

4. The apparatus of claim 1, wherein the exposure threshold for the treatment cycle is associated with an expected area of least radiation exposure on the object to be treated.

5. The apparatus of claim 1, wherein the controller is further configured to calculate the time period based on an expected area of least radiation exposure on the object to be treated, the expected area of least radiation exposure being based on a geometry of the reactor chamber, location of the plurality of EMR emitters within the reactor chamber, and at least one of a location of the holder or the object.

6. The apparatus of claim 1, wherein the holder is configured to transmit about or greater than about 92% of received radiation.

7. The apparatus of claim 1, wherein the holder is a shelf formed of quartz glass.

8. The apparatus of claim 1, wherein the plurality of EMR emitters are doped mercury bulbs.

9. The apparatus of claim 1, wherein each sensor from the plurality of sensors includes an amplifier configured to boost a signal of the applicable sensor and limit interference along a path of the signal.

10. The apparatus of claim 1, wherein the first sensor is disposed equal to or less than about two inches from the first EMR emitter and the second sensor is disposed equal to or less than about two inches from the second EMR emitter.

11. The apparatus of claim 1, wherein each EMR emitter from the plurality of EMR emitters is mounted substantially horizontally within the interior chamber.

12. The apparatus of claim 1, wherein the predefined radiation exposure threshold is at least about 51,000 microwatt/cm².

13. The apparatus of claim 1, wherein the controller is configured to deactivate the plurality of EMR emitters based on the completion of the treatment cycle.

14. The apparatus of claim 1, wherein the controller is configured to deactivate the plurality of EMR emitters based on the access door transitioning away from the closed configuration.

15. The apparatus of claim 1, wherein the EMR emitters and the holder are collectively arranged such that all surfaces of the object supported by the holder that are exposed to the radiation are exposed simultaneously.

16. The apparatus of claim 1, wherein the plurality of sensors is disposed within the housing but outside the reactor chamber and communicatively coupled to the reactor chamber such that the plurality of sensors does not disrupt radiation conveyed within the reactor chamber.

17. The apparatus of claim 1, wherein the first sensor is arranged to not receive any radiation emitted by the second EMR emitter, and the second sensor is arranged to not receive any radiation emitted by the first EMR emitter.

18. The apparatus of claim 1, wherein the first sensor is disposed outside the reactor chamber and communicatively coupled to the reactor chamber via a first lumen extending from adjacent the first sensor to an interior surface of the reactor chamber such that first pass radiation emitted by the first EMR emitter reaches the first sensor via the first lumen,
the second sensor is disposed outside the reactor chamber and communicatively coupled to the reactor chamber via a second lumen extending from adjacent the first sensor to an interior surface of the reactor chamber such that first pass radiation emitted by the second EMR emitter reaches the second sensor via the second lumen.

19. The apparatus of claim 1, wherein the electromagnetic radiation is ultraviolet-C (UV-C) radiation.

20. A method to determine a time period to operate a radiation treatment cycle for an object disposed within a reactor chamber of a housing, the reactor chamber including a plurality of electromagnetic radiation (EMR) emitters configured to emit radiation to treat the object, the housing including a plurality of sensors configured to measure radiation intensity, the method comprising:
receiving from a first sensor from the plurality of sensors a radiation intensity value representative of first pass radiation emitted within the reactor chamber by a first EMR emitter from the plurality of EMR emitters;
receiving from a second sensor from the plurality of sensors a radiation intensity value representative of first pass radiation emitted within the reactor chamber by a second EMR emitter from the plurality of EMR emitters;
identifying a lowest output EMR emitter from the plurality of EMR emitters based on the lesser of the radiation intensity value received from the first sensor and the radiation intensity value received from the second sensor to define a minimum collective output for the plurality of EMR emitters; and
calculating a time period to deliver a predefined minimum radiation exposure threshold of radiation to an area on the object expected to receive the least radiation exposure per unit time relative to the remainder of the object, based on (1) the minimum collective output, (2) a distance associated with first pass radiation provided by one EMR emitter from the plurality of EMR emitters, and (3) a distance and reflectivity associated with second pass radiation provided by one EMR emitter from the plurality of EMR emitters.

21. The method of claim 20, wherein the area on the object is a top face of the object.

22. The method of claim 20, wherein the distance associated with first pass radiation provided by the one EMR emitter is the greater of (1) a distance across which the first EMR emitter delivers first pass radiation to the area, and (2) a distance across which the second EMR emitter delivers first pass radiation to the area.

23. The method of claim 20, wherein the distance associated with second pass radiation provided by the one EMR emitter is the greater of (1) a distance across which the first EMR emitter delivers second pass radiation to the area, and (2) a distance across which the second EMR emitter delivers second pass radiation to the area,
the reflectivity factor associated with the second pass radiation accounting for a reduction in radiation intensity in response to the second pass radiation bouncing off a surface of the reactor chamber.

24. The method of claim 20, wherein the reflectivity accounts for interior surfaces of the reactor chamber to reflect greater than about 85% of radiation received on the surfaces.

25. An apparatus, comprising:
one of more memories; and
one or more processors operatively coupled to the one or more memories, the one or more processors configured to:
receive from a first sensor from a plurality of sensors a radiation intensity value representative of first pass radiation emitted within a reactor chamber by a first EMR emitter from the plurality of EMR emitters;
receive from a second sensor from the plurality of sensors a radiation intensity value representative of first pass radiation emitted within the reactor chamber by a second EMR emitter from the plurality of EMR emitters;
identify a lowest output EMR emitter from the plurality of EMR emitters based on the lesser of the radiation intensity value received from the first sensor and the radiation intensity value received from the second sensor to define a minimum collective output for the plurality of EMR emitters; and
calculate a time period to deliver a predefined minimum radiation exposure threshold of radiation to an area on the object expected to receive the least radiation exposure per unit time relative to the remainder of the object, based on (1) the minimum collective output, (2) a distance associated with first pass radiation provided by one EMR emitter from the plurality of EMR emitters, and (3) a distance and reflectivity associated with second pass radiation provided by one EMR emitter from the plurality of EMR emitters.
